(19) United States
(12) Patent Application Publication
(10) Pub. No.: US 2026/0325027 A1
(43) Pub. Date: Oct. 22, 2026

(54) ATTENTION-INDUCED PHYSIOLOGICAL RESPONSE PREDICTION

(71) Applicant: Apple Inc., Cupertino, CA (US)

(72) Inventors: Brian Pasley, Berkeley, CA (US); Grant H. Mulliken, Los Gatos, CA (US); Hyun Jong Won, Sunnyvale, CA (US); Hope Lutwak, San Francisco, CA (US)

(21) Appl. No.: 19/560,443

(22) Filed: Mar. 9, 2026

Related U.S. Application Data
(60) Provisional application No. 63/772,034, filed on Mar. 14, 2025.

(51) Int. Cl. A61B 5/16; A61B 5/00

(52) U.S. Cl. CPC A61B 5/165; A61B 5/163; A61B 5/486; A61B 5/7435

(57) ABSTRACT

Various implementations disclosed herein include devices, systems, and methods that determine an attention-induced physiological response during presentation of content. For example, an example process may include displaying content that includes an interaction element with a plurality of regions having different illumination characteristics. The process may further include providing instructions to focus on a particular region that is associated with a particular illumination characteristic. The process may further include obtaining a first set of physiological data that is associated with pupillary data. The process may further include determining, based on the first set of physiological data, an attention-induced physiological response during presentation of content corresponding to an interaction with the interaction element. A process may be configured to detect user interactions of a particular type based on detecting occurrences of attention-induced physiological responses by the user that are similar to the attention-induced physiological response.

### CROSS-REFERENCE TO RELATED APPLICATIONS

This Application claims the benefit of U.S. Provisional Application Ser. No. 63/772,034 filed Mar. 14, 2025, which is incorporated herein in its entirety.

### TECHNICAL FIELD

The present disclosure generally relates to presenting content via electronic devices, and in particular, to systems, methods, and devices that determine an interaction event during and/or based on the presentation of electronic content and physiological data.

### BACKGROUND

Determining a user's intent while viewing content on an electronic device can facilitate a more meaningful experience. For example, a user interface element (e.g., a selectable icon or button) may be automatically selected based on determining the user's intent to make such a selection and without the user necessarily having to perform a gesture, mouse click, or other input-device-based action to initiate the selection. Improved techniques for assessing the intent of users viewing and interacting with content may enhance the users'enjoyment, comprehension, and learning of the content. Content creators and systems may be able to provide better and more tailored user experiences based determining user intent to interact with user interface elements.

### SUMMARY

Various implementations disclosed herein include devices, systems, and methods that assess pupillary data (e.g., attention-induced pupil constriction) to predict a user interaction event (e.g., predicting an intention to click) on a particular user interface element (e.g., predicting when a user is focused on a particular portion of the content). In other words, detecting a click intention only using eye/pupil actions (e.g., pupillary data), and not requiring an input device or hand interactions (e.g., a pinch-based movement). For example, a method may identify that, during a particular segment of an experience, a user may be instructed to focus on one or more illumination characteristics of an interaction element that be used to predict an interaction event. For example, a user may direct his or her attention to a bright feature in an icon or other user interface element in order to initiate a “click” or other interaction. In other words, train a user to attend to stimulate the particular pupillary signal's to initiate a click (e.g., “focus on imagining a click with your eye gaze/focus”) in order to replace pinch and enable hands free use, e.g., while wearing a head mounted device (HMD) during an extended reality (XR) experience.

In some implementations, other physiological data may be used to determine an interaction event (e.g., electroencephalography (EEG) data, gaze(s), head/body movement data, and the like). Additionally, determining the user's eye characteristics may involve obtaining images of the eye or electrooculography (EOG) data, microsaccades, and/or head movements, from which pupil response/gaze direction/movement can be determined. For example, some implementations may identify that the user's eye characteristics (e.g., blink rate, stable gaze direction, saccade amplitude/velocity, and/or pupil radius) relate to an interaction with a presentation of an interaction element (e.g., an icon) based on a user's focus upon different regions of the interaction element that have different illumination characteristics. For example, the illumination features may include relatively dark or bright regions. For example, a method may identify that, during a particular segment of the experience, the user's gaze characteristics (e.g., pupil dilation vs. constriction, stable gaze direction and/or velocity) corresponds to a user focusing on a particular icon or user interface element.

In some implementations, determining an interaction event may be based on a characteristic of an environment of the user (e.g., real-world physical environment, a virtual environment, or a combination of each). The device (e.g., a handheld, laptop, desktop, or HMD) provides an experience (e.g., a visual and/or auditory experience) of the real-world physical environment or an XR environment. The device obtains, with one or more sensors, physiological data (e.g., EEG amplitude, electromyography (EMG), pupil modulation, eye gaze saccades, head movements measured by an inertial measurement unit (IMU), etc.) associated with the user. Based on the obtained physiological data, the techniques described herein can determine an interaction event during the experience. Based on the physiological data and associated physiological response (e.g., a user focusing on a particular region of the content), the techniques can provide a response to the user based on the interaction event and adjust the content corresponding to the experience.

Physiological response data, such as EEG amplitude/frequency, pupil modulation, eye gaze saccades, etc., can depend on the individual, characteristics of the scene in front of him or her (e.g., video content), and attributes of the physical environment surrounding the user including the activity/movement of the user. Physiological response data can be obtained while using a device with eye tracking technology (and other physiologic sensors) while users perform tasks. In some implementations, physiological response data can be obtained using other sensors, such as EEG sensors, EMG sensors, EDA sensors, and the like, that may obtain physiological signals from the head, limbs, or other portions of a person. Observing repeated measures of physiological response data to an experience can give insights about the intent of the user.

Several different experiences can utilize the techniques described herein regarding assessing interaction events using a cognitive based user interface. For example, the method can be provided to support users who want to interact with user interface elements using neurological signals without using hands, voice, or overt eye movements like dwell time. Additionally, determining interaction events can be used as an accessibility feature, for example, that enables paralyzed users to interact by selecting computer graphic icons using neurological signals (e.g., think of making a pinch movement for a particular icon) and/or eye gaze data. Additionally, determining interaction events can be used in general applications (e.g., a user interface selection tool, a device wake-up signal, etc.), and might be combined with other eye or touch-based mechanisms, such as to improve signal-to-noise ratio (SNR), robustness, response time, and the like. The cognitive based user interface may utilize two learning systems: i) learn to more accurately detect the neural signal over time, and ii) train the user to produce clearer neural signals (e.g., enhance the accuracy and sensitivity of a neural pinch signal for each user).

Context may additionally be used to determine interaction events. For example, a scene analysis of an experience can determine a scene understanding of the visual and/or auditory attributes associated with content being presented to the user (e.g., what is being presented in video content) and/or attributes associated with the environment of the user (e.g., where is the user, what is the user doing, what objects are nearby). These attributes of both the presented content and environment of the user can improve the determination of the user's intent regarding an interaction event.

Some implementations focus on improving the accuracy for assessing interaction events based on a user's neurological signals, pupillary response, body/head movement data, and the like, by incorporating practice exercises. For example, a machine learning algorithm may be implemented to determine whether or not a user's focus/intent means that he or she is intending to select a particular icon (e.g., attention-induced physiological response prediction).

Some implementations assess physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user should have the ability to access and otherwise find out anything that the system has collected or determined about him or her.

In general, one innovative aspect of the subject matter described in this specification can be embodied in methods, at a device including a processor, that include the actions of displaying, via a user interface, content that includes an interaction element, wherein the interaction element includes a plurality of regions, each region having different illumination characteristics. The actions may further include providing instructions to focus on a particular region of the plurality of regions, the particular region being associated with a particular illumination characteristic. The actions may further include obtaining a first set of physiological data for a first period of time, the physiological data associated with pupillary data. The actions may further include determining, based on the first set of physiological data, an attention-induced physiological response during presentation of the content for the first period of time corresponding to an interaction with the interaction element, wherein a process is configured to detect user interactions of a particular type based on detecting occurrences of attention-induced physiological responses by the user that are similar to the attention-induced physiological response

These and other embodiments can each optionally include one or more of the following features.

In some embodiments, the actions may further include updating a pupillary sensitivity level of the user interface based on the attention-induced physiological response. In some embodiments, the actions may further include obtaining a second set of physiological data for a second period of time, the physiological data associated with pupillary data, determining, based on the second set of physiological data, a second attention-induced physiological response during presentation of the content for the second period of time corresponding to an interaction with the interaction element, and updating the pupillary sensitivity level of the user interface based on the second attention-induced physiological response.

In some embodiments, the process is configured to detect user interactions of the particular type includes predicting that the user is thinking of performing an act based on recognizing that a pattern exhibited in the pupillary data of the physiological data is indicative of the user thinking of performing the act.

In some embodiments, the actions may further include determining user interaction feedback data corresponding to the interaction with the interaction element based on the attention-induced physiological response. In some embodiments, the user interaction feedback data includes providing instructions to the user via the user interface to initiate the attention-induced physiological response corresponding to an interaction event with the interaction element while thinking of performing an act.

In some embodiments, the interaction with the interaction element is classified using a machine learning technique based on the attention-induced physiological response and the different illumination characteristics of the regions.

In some embodiments, the different illumination characteristics of the interaction element includes one or more dark regions and one or more bright regions. In some embodiments, determining an attention-induced physiological response includes determining scene-induced pupil response variation characteristics for the regions of the interaction element, and determining the interaction event of the user during the presentation of content based on the scene-induced pupil response variation characteristics for the regions of the interaction element. In some embodiments, the different illumination characteristics of the interaction element includes one or more dark regions and one or more bright regions.

In some embodiments, the actions may further include modifying the content via the user interface in response to determining the attention-induced physiological response corresponding to the interaction with the interaction element. In some embodiments, the attention-induced physiological response includes a pupillary response.

In some embodiments, the pupillary response is a direction of the pupillary response, a velocity of the pupillary response, or pupillary fixations.

In some embodiments, determining the attention-induced physiological response is based on a combination of neurological signals corresponding to a neurological response and pupillary data corresponding to a pupillary response.

In some embodiments, the device is a head-mounted device (HMD). In some embodiments, the presentation of content is an extended reality (XR) experience that is presented to the user.

In general, one innovative aspect of the subject matter described in this specification can be embodied in methods, at a device including a processor, that include the actions of obtaining physiological data associated with pupil dilation during presentation of content to a user at the device, wherein the content includes an interaction element. The actions may further include predicting, based on the physiological data, that the user is exhibiting an attention-induced pupil diameter pattern, wherein the attention-induced pupil dilation pattern is associated with a particular type of interaction, wherein the attention-induced pupil dilation pattern corresponds to the user directing focus to a particular portion of the interaction element, wherein the particular portion of the interaction element includes different characteristics than other portions of the interaction element. The actions may further include determining user interaction feedback corresponding to an interaction event associated with the interaction element based on the predicting that the user is exhibiting the attention-induced pupil dilation pattern.

These and other embodiments can each optionally include one or more of the following features.

In some embodiments, the predicting is based on interpreting that the attention-induced pupil dilation pattern using a process configured to identify pupil dilation patterns associated with focusing on user interface elements that exhibit different characteristics.

In some embodiments, the actions may further include, and in response to determining that an act associated with the particular type of interaction is performed obtaining additional physiological data associated with neurological signals, and updating a prediction model associated with the user performing the act associated with the particular type of interaction based on the additional physiological data.

In some embodiments, the process configured to detect user interactions of the particular type includes predicting that the user is thinking of performing an act based on recognizing that a pattern exhibited in the physiological data is indicative of the user thinking of performing the act.

In some embodiments, the actions may further include determining user interaction feedback data corresponding to the interaction with the interaction element based on the attention-induced physiological response. In some embodiments, the user interaction feedback data includes providing instructions to the user via the user interface to initiate the attention-induced physiological response corresponding to an interaction event with the interaction element while thinking of performing the act.

In some embodiments, the interaction with the particular portion of the interaction element is classified using a machine learning technique based on the attention-induced physiological response and the different illumination characteristics of the regions. In some embodiments, the different illumination characteristics of the interaction element includes one or more dark regions and one or more bright regions.

In some embodiments, determining an attention-induced physiological response includes determining scene-induced pupil response variation characteristics for the regions of the interaction element, and determining the interaction event of the user during the presentation of content based on the scene-induced pupil response variation characteristics for the regions of the interaction element.

In some embodiments, the actions may further include modifying the content via the user interface in response to determining the attention-induced physiological response corresponding to the interaction with the interaction element. In some embodiments, the attention-induced physiological response includes a pupillary response. In some embodiments, the pupillary response is a direction of the pupillary response, a velocity of the pupillary response, or pupillary fixations. In some embodiments, determining the attention-induced physiological response is based on a combination of neurological signals corresponding to a neurological response and pupillary data corresponding to a pupillary response.

In some embodiments, the device is a head-mounted device (HMD). In some embodiments, the presentation of content is an extended reality (XR) experience that is presented to the user.

In accordance with some implementations, a non-transitory computer readable storage medium has stored therein instructions that are computer-executable to perform or cause performance of any of the methods described herein. In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an environment in which extended reality (XR) content is provided to one or more users wearing head mounted displays (HMDs) in accordance with some implementations.

FIG. 2 illustrates detecting an interaction event while viewing content based on physiological data in accordance with some implementations.

FIGS. 3A, 3B, 3C, and 3D illustrate example interaction elements that include regions having different illumination characteristics in accordance with some implementations.

FIG. 4 illustrates an example interaction element that changes based on user interaction and includes regions having different illumination characteristics in accordance with some implementations.

FIGS. 5A and 5B illustrate detecting an interaction event with a cognitive based user interface based on physiological data in accordance with some implementations.

FIGS. 6A and 6B illustrate detecting an interaction event with a cognitive based user interface based on pupillary data in accordance with some implementations.

FIG. 7 is a chart illustrating averaged pupil response time of each participant based on voluntary feature attention by the participants in accordance with some implementations.

FIG. 8A is a chart illustrating pupil diameter change associated with a user interaction event in accordance with some implementations.

FIG. 8B is a chart illustrating an adaptive threshold sensitivity based on pupillary data in accordance with some implementations.

FIG. 9 illustrates a system diagram for determining an attention-induced physiological response corresponding to an interaction with an interaction element in accordance with some implementations.

FIG. 10 is a flowchart representation of a method for determining an attention-induced physiological response corresponding to an interaction with an interaction element in accordance with some implementations.

FIG. 11 is a flowchart representation of a method for determining user interaction feedback corresponding to an interaction event based on predicting that the user is exhibiting an attention-induced pupil dilation pattern in accordance with some implementations.

FIG. 12 illustrates a system flow diagram of an example process for performing instructions associated with an attention-induced physiological response instruction set, in accordance with some implementations.

FIG. 13 illustrates device components of an exemplary device in accordance with some implementations.

FIG. 14 illustrates an example head-mounted device (HMD) in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

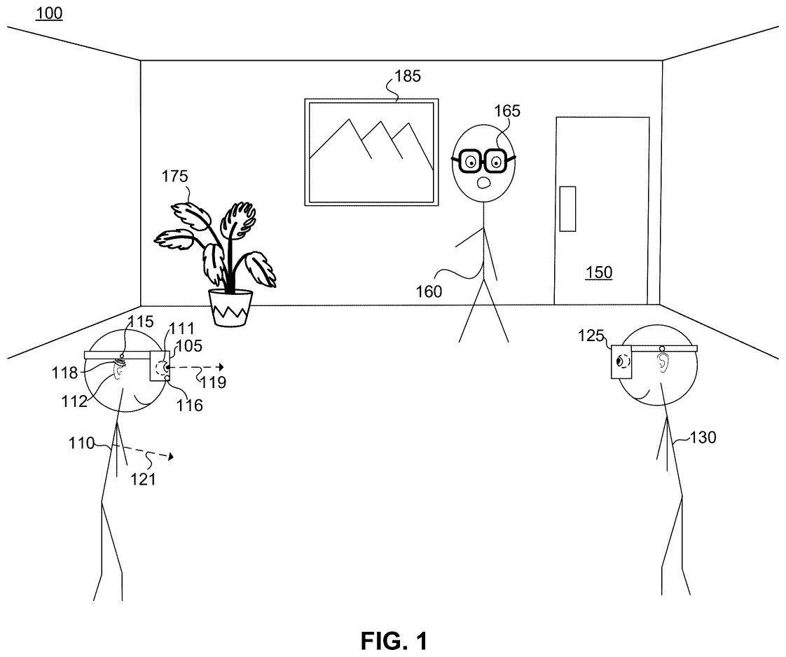

FIG. 1 illustrates a real-world physical environment 100 including a first user 110 wearing a first device 105, a second user 130 wearing a second device 125, a third user 160 wearing a third device 165, a wall-hung picture 185, a plant 175, and a door 150. In some implementations, one or more of the devices 105, 125, 165 is configured to provide content based on one or more sensors on the respective devices or to share information and/or sensor data with one another. In some implementations, one or more of the devices 105, 125, 165 provide content that provides augmentations in XR using sensor data. The sensor data may be used to understand that a user's state is associated with providing user assistance, e.g., a user's appearance or behavior or an understanding of the environment may be used to recognize a need or desire for assistance.

In the example of FIG. 1, the first device 105 includes one or more sensors 116 that capture light-intensity images, depth sensor images, audio data or other information about the user 110 and the physical environment 100. For example, the one or more sensors 116 may capture images of the user's forehead, eyebrows, eyes, eye lids, cheeks, nose, lips, chin, face, head, hands, wrists, arms, shoulders, torso, legs, or other body portion. Sensor data about a user's eye 111, as one example, may be indicative of various user characteristics, e.g., the user's gaze direction 119 over time, user saccadic behavior over time, user eye dilation behavior over time, etc. The one or more sensors 116 may capture audio information including the user's speech and other user-made sounds as well as sounds within the physical environment 100.

One or more sensors, such as one or more sensors 115 on device 105, may identify user information based on proximity or contact with a portion of the user 110. As example, the one or more sensors 115 may capture sensor data that may provide biological information relating to a user's cardiovascular state (e.g., pulse), body temperature, breathing rate, etc.

The one or more sensors 116 or the one or more sensors 115 may capture data from which a user orientation 121 within the physical environment can be determined. In this example, the user orientation 121 corresponds to a direction that a torso of the user 110 is facing.

In some implementations, the content provided by the device 105 and sensor features of device 105 may be provided using components, sensors, or software modules that are sufficiently small in size and efficient with respect to power consumption and usage to fit and otherwise be used in lightweight, battery-powered, wearable products such as wireless ear buds or other ear-mounted devices or head mounted devices (HMDs) such as smart/augmented reality (AR) glasses. Features can be facilitated using a combination of multiple devices. For example, a smart phone (connected wirelessly and interoperating with wearable device(s)) may provide computational resources, connections to cloud or internet services, location services, etc.

In some implementations, data is shared amongst a group of devices to improve user state or environment understanding. For example, device 125 may share information (e.g., images, audio, or other sensor data) corresponding to user 110 or the physical environment 100 (including information about user 130 or user 160) with device 105 so that device 105 can better understand user 110 and physical environment 100.

In some implementations, devices 105, 125, 165 are head mounted devices (HMDs) that present visual or audio content (e.g., extended reality XR content) or have sensors that obtain sensor data (e.g., visual data, sound data, depth data, ambient lighting data, etc.) about the environment 100 or sensor data (e.g., visual data, sound data, depth data, physiological data, etc.) about the users 110, 130, 160. Such information may, subject to user authorizations, permissions, and preferences, be shared amongst the device 105, 125, 165 to enhance the user's experiences on such devices.

In some implementations, the devices 105, 125, 165 obtain physiological data (e.g., EEG amplitude/frequency, pupil modulation, eye gaze saccades, etc.) from the users 110, 130, 160 via one or more sensors that are proximate or in contact with the respective user 110, 130, 160. For example, the device 105 may obtain pupillary data (e.g., eye gaze characteristic data) from an inward facing eye tracking sensor. In some implementations, the devices 105, 125, 165 include additional sensors for obtaining image or other sensor data of the physical environment 100.

In some implementations, the devices 105, 125, 165 are wearable devices such as ear-mounted speaker/microphone devices (e.g., headphones, ear pods, etc.), smart watches, smart bracelets, smart rings, smart/AR glasses, or other head-mounted devices (HMDs). In some implementations, the devices 105, 125, 165 are handheld electronic devices (e.g., smartphones or tablets). In some implementations, the devices 105, 125, 165 are laptop computers or desktop computers. In some implementations, the devices 105, 125, 165 have input devices such as audio command input systems, gesture recognition-based input systems, touchpads or touch-sensitive displays (also known as a “touch screen” or “touch screen display”). In some implementations, multiple devices are used together to provide various features. For example, a smart phone (connected wirelessly and interoperating with wearable device(s)) may provide computational resources, connections to cloud or internet services, location services, etc.

FIG. 1 illustrates an example in which the devices within the physical environment 100 include HMD devices 105, 125, 165. Numerous other types of devices may be used including mobile devices, tablet devices, wearable devices, hand-held devices, personal assistant devices, AI-assistant-based devices, smart speakers, desktop computing devices, menu devices, cash register devices, vending machine devices, juke box devices, or numerous other devices capable of presenting content, capturing sensor data, or communicating with other devices within a system, e.g., via wireless communication. For example, assistance may be provided to a vision impaired person to help the person understand a menu by providing data from the menu to a device being worn by the vision impaired person, e.g., enabling that device to enhance the user's understanding of the menu by providing visual annotations, audible cues, etc.

In some implementations, the devices 105, 125, 165 include eye tracking systems for detecting eye position and eye movements. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user. Moreover, an illumination source on a device may emit NIR light to illuminate the eyes of the user and the NIR camera may capture images of the eyes of the user. In some implementations, images captured by the eye tracking system may be analyzed to detect position and movements of the eyes of the user, or to detect other information about the eyes such as pupil dilation or pupil diameter. Moreover, the point of gaze estimated from the eye tracking images may enable gaze-based interaction with content shown the device. Additional cameras may be included to capture other areas of the user (e.g., an HMD with a jaw cam to view the user's mouth, a down cam to view the body, an eye cam for tissue around the eye, and the like). These cameras and other sensors can detect motion of the body, or signals of the face modulated by the breathing of the user (e.g., remote PPG).

In some implementations, the devices 105, 125, 165 have graphical user interfaces (GUIs), one or more processors, memory and one or more modules, programs or sets of instructions stored in the memory for performing multiple functions. In some implementations, the users 110, 130, 160 may interact with a GUI through voice commands, finger contacts on a touch-sensitive surface, hand/body gestures, remote control devices, or other user input mechanisms. In some implementations, the functions include viewing/listening to content, image editing, drawing, presenting, word processing, website creating, disk authoring, spreadsheet making, game playing, telephoning, video conferencing, e-mailing, instant messaging, workout support, digital photographing, digital videoing, web browsing, digital music playing, or digital video playing. Executable instructions for performing these functions may be included in a computer readable storage medium or other computer program product configured for execution by one or more processors.

In some implementations, the devices 105, 125, 165 employ various physiological or behavioral sensor, detection, or measurement systems. Detected physiological data may include, but is not limited to, EEG, electrocardiography (ECG), electromyography (EMG), functional near infrared spectroscopy signal (fNIRS), blood pressure, skin conductance, or pupillary response. Detected behavioral data may include, but is not limited to, facial gestures, facial expressions, body gestures, or body language based on image data, voice recognition based on acquired audio signals, etc.

In some implementations, the devices 105, 125, 165 (or other devices) may be communicatively coupled to one or more additional sensors. For example, a sensor (e.g., an EDA sensor) may be communicatively coupled to a device 105, 125, 165 via a wired or wireless connection, and such a sensor may be located on the skin of a user (e.g., on the arm, placed on the hand/fingers of the user, etc.). For example, such a sensor can be utilized for detecting EDA (e.g., skin conductance), heart rate, or other physiological data that utilizes contact with the skin of a user. Moreover, a device 105, 125, 165 (using one or more sensors) may concurrently detect multiple forms of physiological data in order to benefit from synchronous acquisition of physiological data or behavioral data. Moreover, in some implementations, the physiological data or behavioral data represents involuntary data, e.g., responses that are not under conscious control. For example, a pupillary response may represent an involuntary movement. In some implementations, a sensor is placed on the skin as part of a watch device, such as a smart watch.

In some implementations, one or both eyes of a user, including one or both pupils of the user present physiological data in the form of a pupillary response (e.g., eye gaze characteristic data). The pupillary response of the user may result in a varying of the size or diameter of the pupil, via the optic and oculomotor cranial nerve. For example, the pupillary response may include a constriction response (miosis), e.g., a narrowing of the pupil, or a dilation response (mydriasis), e.g., a widening of the pupil. In some implementations, a device may detect patterns of physiological data representing a time-varying pupil diameter. In some implementations, the device may further determine the interpupillary distance (IPD) between a right eye and a left eye of the user.

The user data (e.g., upper facial feature characteristic data, lower facial feature characteristic data, and eye gaze characteristic data, etc.), including information about the position, location, motion, pose, etc., of the head or body of the user, may vary in time and a device 105, 125, 165 (or other devices) may use the user's data to track a user state. In some implementations, the user data includes texture data of the facial features such as eyebrow movement, chin movement, nose movement, cheek movement, etc. For example, when a person (e.g., user 110, 130, 160) performs a facial expression or micro expression associated with lack of familiarity or confusion, the upper and lower facial features can include a plethora of muscle movements that are used to assess the state of the user based on the captured data from sensors.

The physiological data (e.g., eye data, head/body data, etc.) and behavioral data (e.g., voice, facial recognition, etc.) may vary in time and the device may use the physiological data or behavioral data to measure a physiological/behavioral response or the user's attention to object or intention to perform an action. Such information may be used to identify a state of the user with respect to whether the user needs or desires assistance.

Information about such assistance predictions and how a user's own data is used may be provided to a user and the user given the option to opt out of automatic predictions/use of their own data and given the option to manually override assistance features. In some implementations, the system is configured to ensure that users' privacy is protected by requiring permissions to be granted before user state is assessed or assistance is enabled.

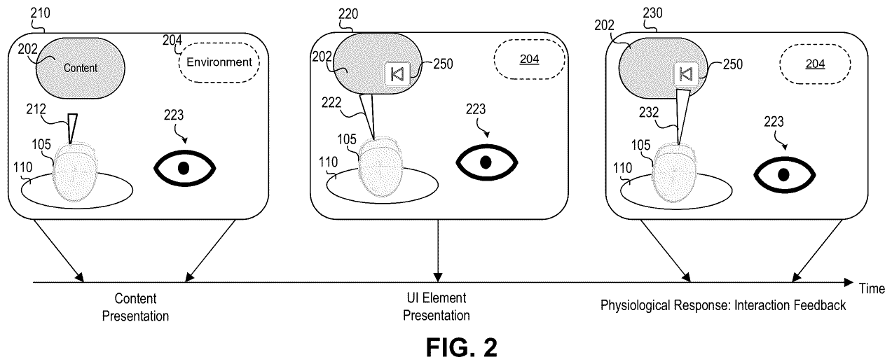

FIG. 2 illustrates assessing whether there is an interaction event while viewing content based on physiological data. In particular, FIG. 2 illustrates a user (e.g., user 110 wearing device 105 of FIG. 1) being presented with content 202 in an environment 204 during a content presentation where the user, via obtained physiological data, has a physiological response to the content (e.g., the user looks towards portions of the content as detected by eye gaze characteristic data). For example, at content presentation instant 210, a user is being presented with content 202 that includes visual content (e.g., a video, virtual reality, augmented reality, and the like), and the user's physiological data such as eye gaze characteristic data 212 and pupillary tracking data 213 may be obtained and tracked as a baseline. Then, at content presentation instant 220, while the user eye gaze characteristic data 222 (pupillary tracking data 223) is engaged (e.g., looking at) content 202, the content 202 presents interactive element 250. After a segment of time after the user's physiological data is analyzed (e.g., by a physiological data instruction set), as illustrated at content presentation instant 230, the user's eye gaze characteristic data 232 and pupillary tracking data 233 is monitored as the user 110 is focused upon the interactive element 250, as the user interacts with the interactive element 250. Therefore, the content 202 may be updated based on the interaction of the user upon the interactive element 250 (e.g., the user wants to select the virtual icon represented by interactive element 250 by attending to the virtual icon). As the user 110 attends to the icon 250, the pupillary data signals are collected and analyzed for determining whether the system can predict the action based only on the pupillary data. In some implementations, other physiological data may be obtained and tracked such as, inter alia, neurological data (EEG data), body/head movement data, and the like.

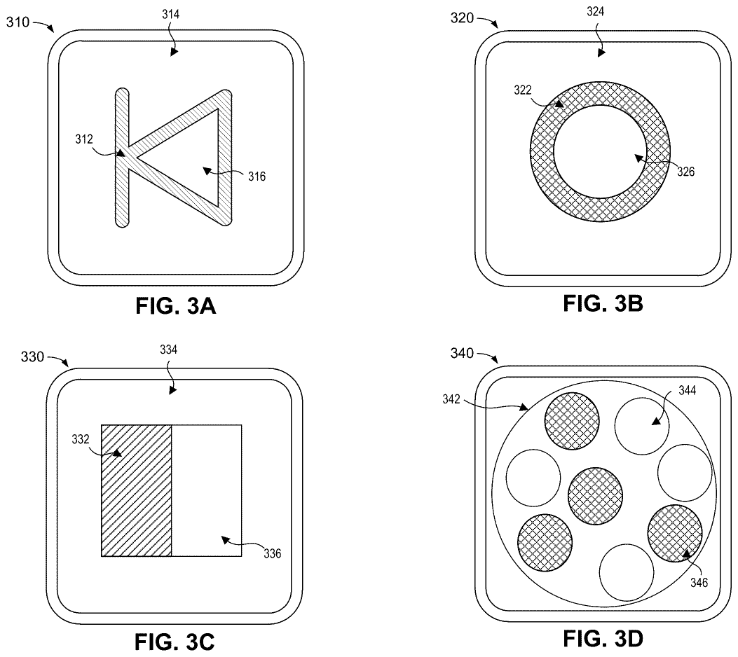

FIGS. 3A, 3B, 3C, and 3D illustrate example interaction elements that include regions having different illumination characteristics in accordance with some implementations. FIG. 3A presents interaction element 310, which is a closer view of the interaction element 350 of FIG. 3 that includes different regions, area 312, area 314, and area 316. Area 312 illustrates a brighter illumination characteristic that forms an example shape (e.g., a triangle and line adjacent to one of the corners). Area 314 illustrates an area with a different illumination characteristic (e.g., may be darker) in comparison to area 312.

FIG. 3B presents interaction element 320 (e.g., concentric circles) that includes different regions, area 322, area 324, and area 326. Area 322 illustrates a brighter illumination characteristic that forms an example shape (e.g., a circle). Area 324 and area 326 (e.g., areas outside and inside area 322, respectively) illustrate areas with different illumination characteristics (e.g., may be darker or lighter) in comparison to area 322.

FIG. 3C presents interaction element 330 (e.g., a square with two different illumination characteristics) that includes different regions, area 332, area 334, and area 336. Area 332 illustrates a brighter illumination characteristic that forms an example shape (e.g., a rectangle). Area 334 (e.g., the other half of the square) and area 336 (e.g., areas outside the square) illustrate areas with different illumination characteristics (e.g., may be darker or lighter) in comparison to area 332.

FIG. 3D presents interaction element 340 (e.g., a circular element 342 that includes eight different areas or circles with different illumination characteristics). For example, the interaction element 340 includes circular element 342 that includes different regions, such as area 344 (a bright white circular element) and area 346 (a darker circular element). Area 344 illustrates a brighter illumination characteristic that forms an example shape (e.g., a circle). Area 346 (e.g., a circle) illustrates an area with different illumination characteristics (e.g., may be darker or lighter, or a different shade) in comparison to area 344 (a bright white circular element).

Although not illustrated as such in FIG. 3A-3D, in some implementations, area 316, for example, the area formed inside of the shape formed by area 312, may also include different illumination characteristics than both area 314 and area 312. For example, if an animation effect is provided for interaction element 310, the area 316 may flash in an alternate pattern with area 312, while area 314 remains constant. As discuss herein, the animation effect may be provided to the user if it is detected that the user has focused his or her attention at the interaction element for a certain period of time (e.g., great than two seconds), then the system may then generate the animation effect to let the user know that in an additional period of time the interaction element will be activated (e.g., the user wants to “click” on the particular icon represented by the interactive element 310). The different illumination characteristics/effects described herein for FIG. 3A, may also occur for any of the other interaction elements 320, 330, and 340.

In some implementations, if the user continuously keeps attending (e.g., “focused”) on a particular area, such as, inter alia, area 312 of the interaction element 310 (e.g., an illuminated portion of the interaction element 310), or area 344 of the interaction element 340 (e.g., a bright circle), etc., then animation effects may initially begin on the interaction element 310 or 340, to indicate to the user that if they keep focusing on that area of the interaction element, it will be selected or an action will occur (e.g., the user is clicking on a virtual icon based on their pupillary response). For example, the pupil constricts during attention to a bright stimulus feature. In some implementations, if interaction element 340 starts to change (e.g., become animated such as moves/shakes, changes in visual appearance, etc.) then the user may know to look away unless the user wants that icon selected. Thus, a user has to “dwell” a certain amount of time to be selected/clicked. For example, after some amount of time, such as a first interaction threshold (e.g., two seconds of focusing on area 344 of interactive element 340, a bright circle), the interactive element 340 changes/animates, and if a user continues to look at area 344 of interactive element 340, after a longer amount of time such as a second interaction threshold (e.g., an additional two seconds), then interactive element 340 may be selected.

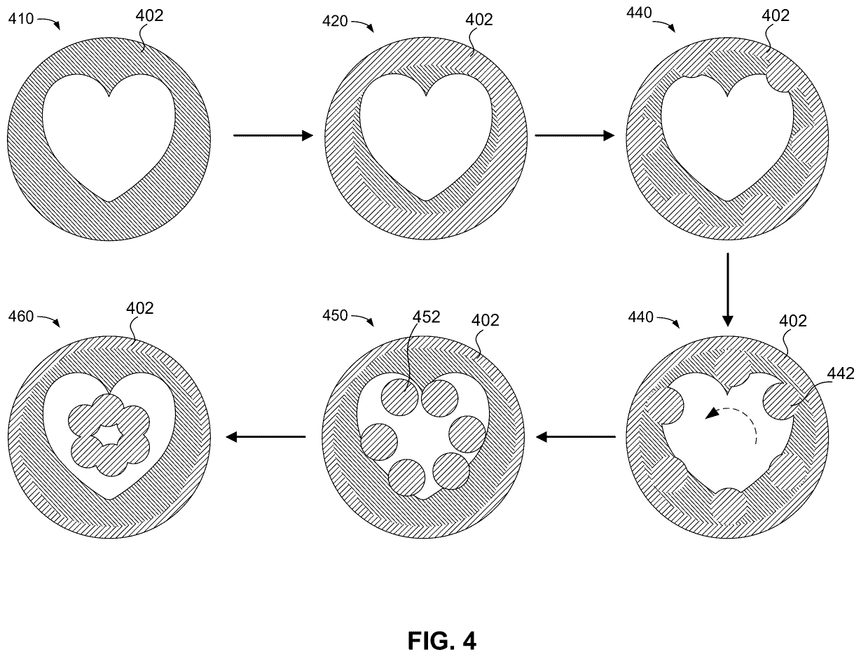

FIG. 4 illustrates an example interaction element 402 that changes based on user interaction and includes regions having different illumination characteristics in accordance with some implementations. In particular, FIG. 4 illustrates the interaction element 402 over different period times (e.g., time periods 410, 420, 430, 440, 450, 460) that includes alterations that may further elicit attention-induced pupil constrictions.

In some implementations, during time period 410, the interaction element 402 is a static image that includes a heart inside of a circle. For example, the interaction element 402 may remain a static image (e.g., a stationary icon on a user interface) until there is some type of user interaction with the interaction element 402. Then, during an indication of some type of action (e.g., pupillary data/gaze towards the element, a mouse hover, etc.), the remaining time periods (e.g., time periods 420, 430, 440, 450, 460) illustrate alterations that may occur over time while the action continues (e.g., the user constantly/consistently is looking at the interaction element 402. For example, at time period 420, some of the illuminations characteristics start to change, at time period 430, dots start to form outside of the heart but inside of the outer circle edge. Then between time periods 440 and 460, the dots begin to move to inside the heart and center within the heart as they spin around the inside of the interaction element 402 in a counterclockwise motion. In some implementations, the dots have different illumination characteristics, similar to the interactive element 340 of FIG. 3D (e.g., bright and dark circles).

In some implementations, the use of the interaction element 402 improves the stimulus itself in order to more efficiently elicit attention-induced pupil constrictions. In some implementations, a stimulus that participants may be instructed to attend to to train an attention algorithm was a static image (e.g., interaction elements 310, 320, 330, and/or 340 of FIG. 3A-3D, respectively). However, trying to click by solely focusing on white dots in a static image may sometimes be difficult. Thus, interaction element 402 includes other features that can be altered from a static image in order to help participants voluntarily elicit pupil constrictions by attending to the white dots in the prompt. For example, to create a multi modal stimulus, interaction element 402 includes other features to more naturally elicit pupil constrictions, such as features like motion, higher spatial frequency content, and/or implied depth, that may independently bolster pupil response when attending to different illumination characteristics (e.g., luminance values). Thus, as illustrated by FIG. 4 for interaction element 402, over time (e.g., multiple frames of content, such as a video), motion (e.g., between time period 440 and time period 450, the dots start turning counterclockwise), higher spatial frequency (e.g., the crispness of each dot), and implied depth (e.g., brighter dots occlude darker dots) are included as part of the interaction element 402. Additionally, in some implementations, these additional features may add another signal that can be measured like naturalistic eye tracking movements which may help measure and track a user's cognitive state and improve the attention algorithm discussed herein.

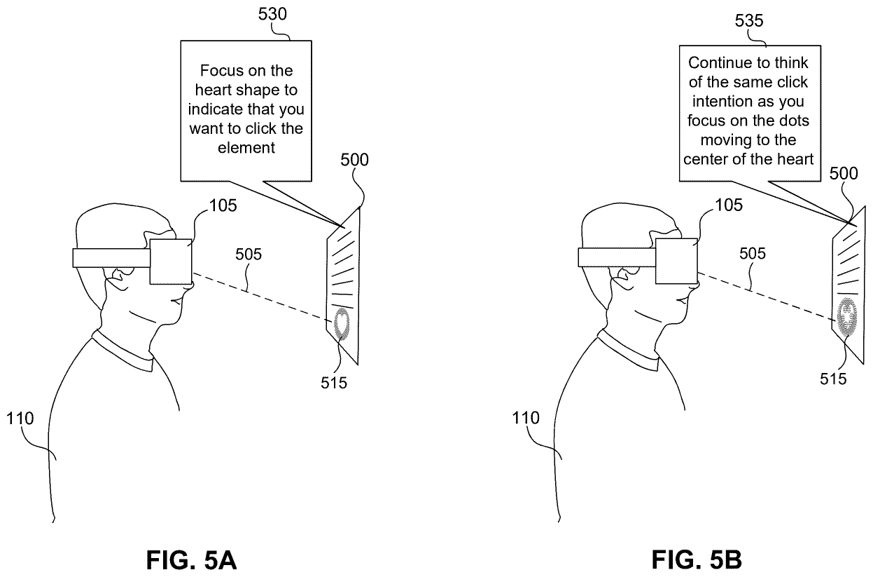

FIGS. 5A and 5B illustrate detecting an interaction event with a cognitive based user interface based on neurological data in accordance with some implementations. FIGS. 5A and 5B illustrate the user 110 wearing device 105, as the user 102 is virtually interacting with a user interface element 515 (e.g., interactive element 402 of FIG. 4) of a user interface 500 while gazing along gaze direction 505. In particular, FIGS. 5A and 5B illustrate an interaction with user interface 500 as the user is facing the user interface 500. In this example, the user 102 is using device 105 to view and interact with an XR environment that includes the user interface 500. In an exemplary embodiment, the physiological data obtained by one or more sensors on the device 105, or communicatively coupled to the device 105. The physiological data may include eye gaze characteristic data, such as pupillary tracking information while the user 110 is gazing along gaze direction 505. Additionally, in some implementations, other physiological data may be obtained by other sensors or from other devices.

The example of FIG. 5A illustrates the user 110 being shown the interaction element 515 on the user interface 500 and the user is instructed to focus on the interaction element 515 as if they are trying to select the element via his or her gaze, as illustrated by the notification 530 (e.g., “Focus on the heart shape to indicate that you want to click the element”). For example, instructing the user to look at and attend to the interaction element 515 and think about clicking the user interface icon with your attention. Additionally, or alternatively, the user may be instructed to focus specifically on the interaction element, e.g., “image white dots getting brighter”, or “look at the button icon when it pops out then imaging pushing it back with your gaze,” in order to induce an action, such as visual attention upon the interaction element.

The example of FIG. 5B illustrates the user 110 being attentive to the interaction element 515 on the user interface 500, and the user is instructed to utilize that same focused attention towards the interaction element 515 as the interaction element 515 is altered, as illustrated by the notification 535 (e.g., “Continue to think of the same click intention as you focus on the dots moving to the center of the heart”). For example, have the user thinking of clicking the icon with the user eye gaze/attention, and/or focus on a particular illumination characteristic(s) on the interaction element 515 (e.g., focus on the white dot). The notifications 530, 535 may be visual notifications, audio notifications, or both visual and audio notifications.

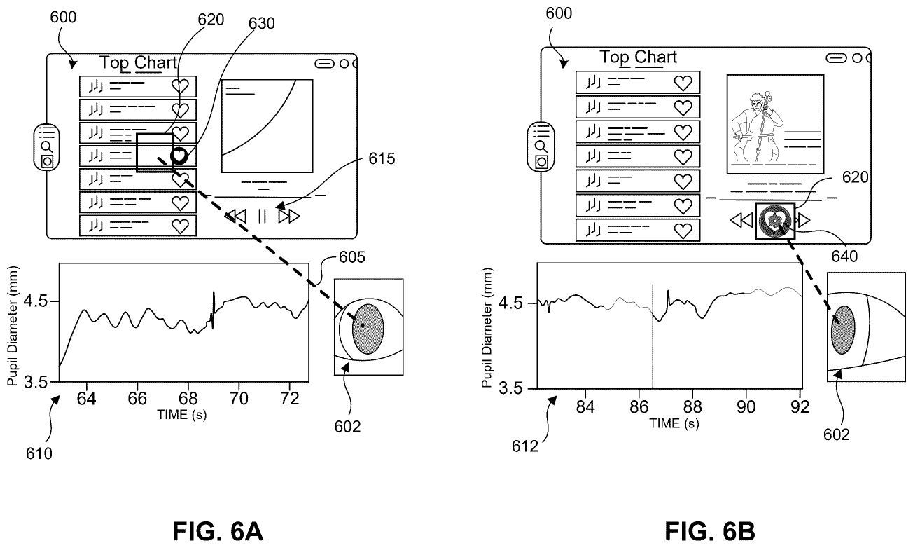

FIGS. 6A and 6B illustrate detecting an interaction event with a cognitive based user interface based on pupillary data using an attention algorithm in accordance with some implementations. FIGS. 6A and 6B illustrate tracking pupillary data of an eye 602 of a user (e.g., user 110 wearing device 105) as the user is virtually interacting with a user interface element (e.g., interactive element 402 of FIG. 4) of a user interface 600 while gazing along gaze direction 605. In particular, FIGS. 6A and 6B illustrate an interaction with user interface 600 as the user is using an application of the user interface 600. In this example, the user is using a device (e.g., wearing an HMD, such as device 105) to view and interact with an XR environment that includes the user interface 600 (e.g., selecting music from music application). In an exemplary embodiment, the physiological data obtained by one or more sensors on the device 105, or communicatively coupled to the device 105. The physiological data may include eye gaze characteristic data, such as pupillary tracking information while the user 110 is gazing along gaze direction 605. Furthermore, the system is tracking the pupillary data for a first period of time, as illustrated by graph 610, and tracking the pupillary data for a second period of time, as illustrated by graph 612.

The example of FIG. 6A illustrates the user is gazing around the user interface 400 and the interaction element 620 (e.g., a square area) follows where the user is gazing towards, but not necessarily focused thereon. In other words, the interaction element 620 provides a cursor like icon (e.g., a square shaped spy glass) that follows the user's gaze as the user gazes upon the user interface 600 along gaze direction 605. Thus, as the user gazes towards the interaction element 630 (e.g., square shaped spy glass of the interaction element 620 intersects with a portion of the interaction element 630), then the interaction element 630 may start to change (e.g., one or more different illumination characteristics may change, or different portions/elements may be moved and/or be added, as illustrated by the circles during the different time periods of FIG. 4).

The example of FIG. 6B illustrates the user is focused on selecting the pause/play button (e.g., area 615 in FIG. 6A). For example, as the user is focused upon the pause/play button, the interaction element 620, after a period of time that the user looks at the pause/play button (e.g., longer than 1 second), displays another interaction element 640 within the same area (e.g., interaction element 402 of FIG. 4). The interaction element 630 (e.g., interaction element 402 of FIG. 4), includes different illumination characteristics that the user may be able to better focus on or was trained to focus on (e.g., “focus on the moving dots to indicate that you want to click the element”) so the system can detect that the user in fact wants to make the selection of that element (e.g., select the pause/play button).

In some implementations, the use of the interaction element 620 (e.g., interaction element 402 of FIG. 4) improves the stimulus itself in order to more efficiently elicit attention-induced pupil constrictions. In some implementations, a stimulus that participants may be instructed to attend to was a static image, but based on participant feedback, trying to click by solely focusing on the white dots in a static image may sometimes be difficult. Thus, there are other features in a static image that may be altered in order to help participants voluntarily elicit pupil constrictions by attending to the white dots in the prompt. For example, to create a multi modal stimulus, other features may be included to more naturally elicit pupil constrictions, such as features like motion, higher spatial frequency content, and/or implied depth, can independently bolster pupil response when attending to luminance. Thus, as illustrated by FIG. 4 for interaction element 402, over time (e.g., multiple frames of content, such as a video), motion, higher spatial frequency (e.g., the crispness of the dots), and implied depth (e.g., the white dots occlude the black dots) are included as part of the interaction element 402. Additionally, in some implementations, these additional features may add another signal that can be measured like naturalistic eye tracking movements which may help measure and track a user's cognitive state and improve the attention algorithm discussed herein.

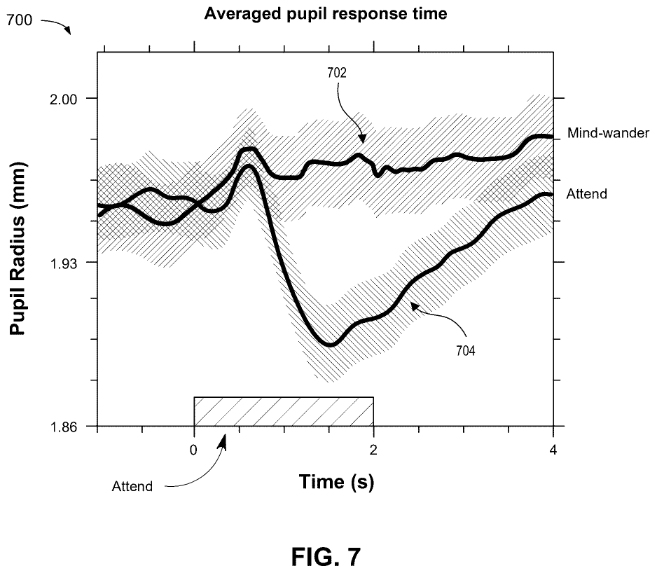

FIG. 7 is an example chart 700 illustrating averaged pupil response time of each participant based on voluntary feature attention by the participants in accordance with some implementations. For example, chart 700 illustrates data averaged over participants showing a robust pupil constriction (red curve) time-locked to the initiation of voluntary feature attention by the participant (right). For example, each participant maybe shown the interaction element 402 of FIG. 4, and are told to focus at the particular area 344 (e.g., brighter element) that includes different illumination characteristics than the area 346 (e.g., darker element). For example, for a first subset of data, the participants are told not to focus on that icon (e.g., interaction element 402), thus each user is “mind wandering”, which is represented by the line 702. Then, for a second subset of data, the participants are told to focus on the illuminated portion of the icon (e.g., area 344 of interaction element 402), thus each user is “focused”, which is represented by the line 704.

In some implementations, the presentation of the interaction element includes pixel information for a plurality of pixels, and determining that the pupillary response corresponds to directing attention to the region of the regions of the interaction element (e.g., area 312 of interaction element 310 of FIG. 3A) includes determining an estimated perceived luminance for each pixel in the region based on the pixel information. For example, the device 105 may collect pixel information from the display and converts the RGB values into an estimated perceived luminance. In some implementations, estimated perceived luminance may be calculated by using a linear formula: Lum=(0.21R+0.72G+0.07B). Additionally, or alternatively, estimated perceived luminance may be calculated by using a nonlinear formula: Lum=√(0.299R²+0.587G²+0.114B²). Moreover, in some implementations, estimated perceived luminance may be determined through feature embeddings via a machine learning model.

In some implementations, determining an interaction event includes determining scene-induced pupil response variation characteristics for the regions of the interaction element, and determining the interaction event during the presentation of the interaction element based on the scene-induced pupil response variation characteristics for the regions of the interaction element. A method may include subtracting from the pupil response the low-level scene-induced pupil response variation as given by the calculated perceived luminance or by the feature embeddings via a machine learning model. For example, when a user's gaze intersects with a user interface element (e.g., area 312 of interaction element 310 of FIG. 3A), a machine learning algorithm predicts “click” or “no click” for each time point, based on the presence of an attention-induced pupil response (e.g., after controlling for the low-level perceived luminance.)

In some implementations, the techniques described herein can utilize a training or calibration sequence to adapt to the specific physiological characteristics of a particular user 110. In some implementations, the techniques present the user 110 with a training scenario in which the user 110 is instructed to interact with on-screen items (e.g., interactive objects). By providing the user 110 with a known intent or area of interest (e.g., via instructions), the techniques can record the user's physiological data (e.g., pupillary data) and identify a pattern associated with the user's physiological data. In some implementations, the techniques can change a visual characteristic (e.g., a feedback mechanism) associated with content in order to further adapt to the unique physiological characteristics of the user 110. For example, the techniques can direct a user to mentally select a button (e.g., an interactive element) associated with an identified area in the center of the screen on the count of three and record the user's physiological data (e.g., pupillary data) to identify a pattern associated with the user's interaction event. Moreover, the techniques can change or alter a visual characteristic associated with the feedback mechanism in order to identify a pattern associated with the user's physiological response to the altered visual characteristic. In some implementations, the pattern associated with the physiological response of the user 110 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying). In some implementations, a “click” threshold may be utilized for training a machine learning model. For example, a “click” threshold may be increased or decreased in real-time to maximize true positive events and minimize false positives. Additionally, or alternatively, in some implementations, implicit feedback from the user (e.g., if a sequence of interactions indicates one interaction was an error) may be used to determine true versus false positives in real-time and the “click” threshold may be adapted by the system accordingly. In some implementations, the techniques described herein can utilize a training process or calibration sequence to involve “gamification”, where the user learns to achieve a certain task over time where there is an animation that corresponds to the real-time output of a machine learning model prediction about the probability of click. For example, controlling and closing a ring animation, where the ring closes in proportion to the model's predicated click probability.

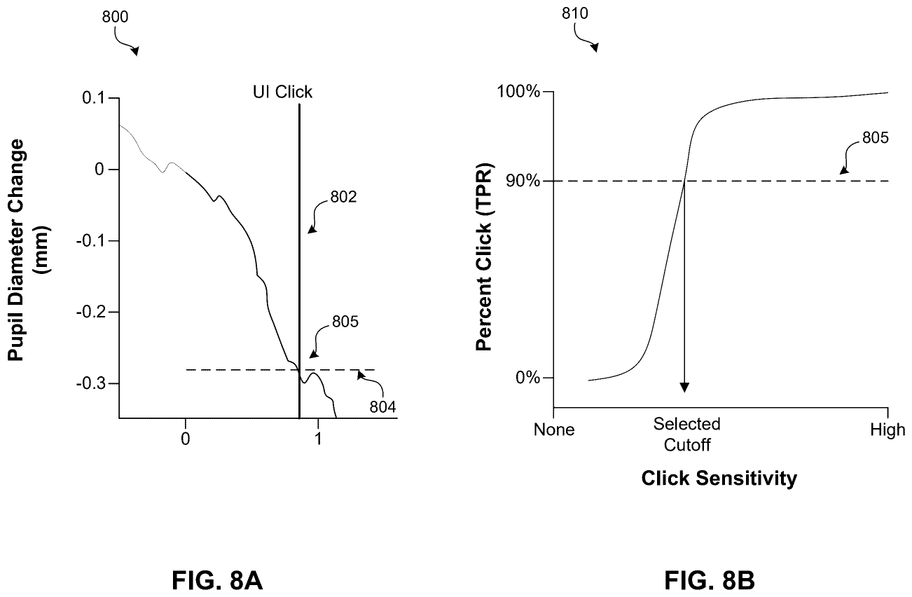

FIG. 8A is an example chart 800 illustrating pupil diameter change associated with a user interaction events in accordance with some implementations. For example, chart 800 illustrates data showing pupil diameter changes over time up until the intended user interface click. In other words, the variability in the pupil constriction reduces as a session progresses for each participant. For example, each participant maybe shown the interaction element 402 of FIG. 4, and are told to focus at a particular area 344 (e.g., moving dots) that includes different illumination characteristics. For example, for a first subset of data, the participants are told not to focus on that icon (e.g., interaction element 402), thus each user is “mind wandering”. Then, for a second subset of data, the participants are told to focus on the illuminated portion of the icon, thus each user is “focused” on clicking the user interaction element to initiate a click (represented by the timeline 802) which intersects the threshold line 804 at the area 805. The threshold line 804 may be adapted per user and is further discussed with reference to FIG. 8B.

FIG. 8B is an example chart 810 illustrating an adaptive threshold sensitivity based on pupillary data in accordance with some implementations. In particular, chart 810 illustrates a click cutoff value that may be varied each trial according to an adaptive procedure (sensitivity value on next trial is set to best estimate of the threshold line 805, e.g., a 90% true positive rate). In some implementations, a calibration procedure terminates when confidence interval of the 90% TPR threshold falls below a specified size. The corresponding threshold may be the calibrated threshold value. In some implementations, possible user feedback may be provided such as audio chimes at each trial indicating how close the confidence interval is to the termination value.

In some implementations, the adaptive threshold may be continuously updated throughout usage of the application by detecting correct/incorrect events and using the adaptive algorithm to determine a new threshold (e.g., updating a pupillary sensitivity level of the user interface based on the attention-induced physiological response). This allows the procedure to stay optimal in the midst of any changes to the user that might change the optimal threshold at any given time (e.g., fatigue, learning, etc). In some implementations, determining the adaptive threshold for an attention algorithm may be based on a Quick Estimation by Sequential Testing (QUEST) algorithm. A QUEST algorithm may be a Bayesian statistical method that is used in psychophysics to efficiently estimate a sensory threshold by dynamically adjusting the stimulus intensity based on the participant's previous responses, essentially learning as the experiment progresses, providing a precise threshold measurement with minimal trials needed. The attention algorithm may use a QUEST algorithm, or similar algorithm, to learn over time a user's sensitivity to a stimulus (e.g., selecting an interactive element via attention).

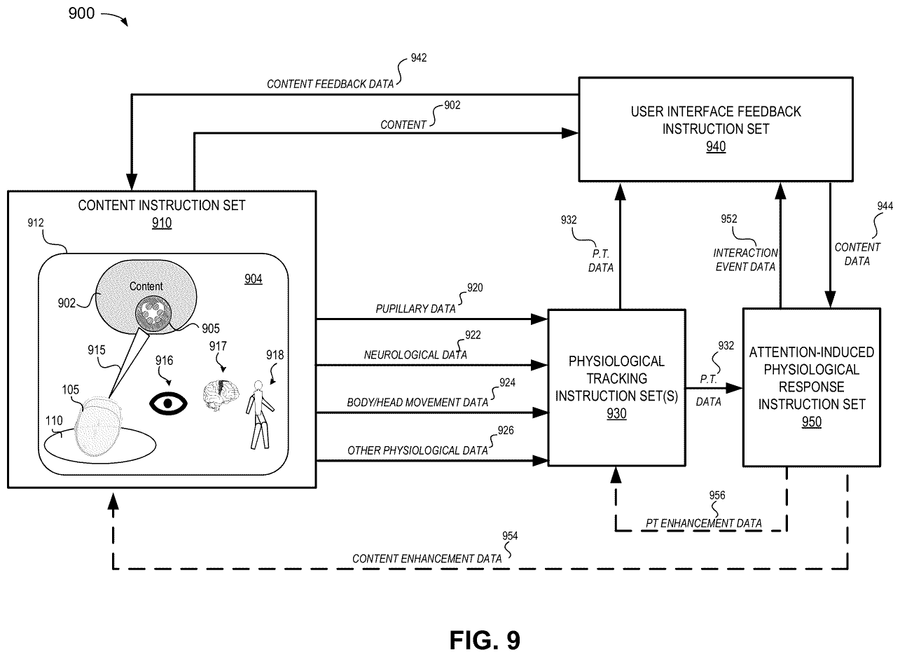

FIG. 9 is a system flow diagram of an example environment 900 in which a attention-induced physiological response prediction system can assess predictions of interaction event based on physiological data (e.g., predicting that the user is thinking of performing an act such as selecting the element via his or her gaze), according to some implementations. In some implementations, the system flow of the example environment 900 is performed on a device (e.g., device 105 of FIG. 1), such as a mobile device, desktop, laptop, or server device. The content of the example environment 900 can be displayed on a device (e.g., device 105 of FIG. 1) that has a screen for displaying images and/or a screen for viewing stereoscopic images such as an HMD. In some implementations, the system flow of the example environment 900 is performed on processing logic, including hardware, firmware, software, or a combination thereof. In some implementations, the system flow of the example environment 900 is performed on a processor executing code stored in a non-transitory computer-readable medium (e.g., a memory).

The system flow of the example environment 900 acquires and presents content 902 (e.g., video content or a series of image data) to user 110, analyzes the content 902 and/or the environment 904 for context data, obtains physiological data associated with the user during presentation of the content, assesses a user's intent to interact with the interaction element 905 (e.g., interaction element 402 of FIG. 4) based on the physiological data of the user (e.g., pupillary signals), and updates the content based on the interaction event (e.g., if the user 110 focusing his or her attention and thinks of performing an act associated with the interaction element 905 (e.g., a pinch) to activate or select the interaction element 905). For example, an attention-induced physiological response prediction technique described herein predicts, based on obtained physiological data (e.g., neurological signals, such as, inter alia, EEG signals), that the user is thinking of performing an act (e.g., selecting an icon with his or her gaze), and determines the user's intent to interact with the interaction element 905 during an experience (e.g., watching a video) by updating the content that is based on the interaction event (e.g., a notification, auditory signal, an alert, and the like, that alerts the user that they have selected the interaction element 905 during the presentation of content 902).

The example environment 900 includes a content instruction set 910 that is configured with instructions executable by a processor to provide and/or track content 902 for display on a device (e.g., device 105 of FIG. 1). For example, the content instruction set 910 provides content presentation instant 912 that includes content 902 to a user 110 while user is within a physical environment 904 (e.g., a room, outside, etc.). For example, content 902 may include background image(s) and sound data (e.g., a video). The content presentation instant 912 could be an XR experience, or content presentation instant 912 could be a MR experience that includes some CGR content and some images of a physical environment. Alternatively, the user could be wearing a HMD and is looking at a real physical environment either via a live camera view, or the HMD allows a user to look through the display, such as wearing smart glasses that user can see through, but still be presented visual and/or audio cues. During an experience, while a user 110 is viewing the content 902, physiological data may be monitored and tracked and sent to the physiological tracking instruction set 930. For example, neurological signal information 917 (e.g., EEG data) of the user's brain activity (e.g., motor cortex activity) can be monitored and sent as neurological data 922, eye gaze characteristic data 915 and pupillary tracking information 916 of the user's eyes can be monitored and sent as pupillary data 920, and body and head tracking information 918 of the user's body movement activity can be monitored and sent as body/head movement data 924. Additionally, other physiological data 926 can be monitored and sent as physiological data 914 such as head movement data obtained from an IMU or image data.

The environment 900 further includes a physiological tracking instruction set 930 to track a user's physiological attributes as physiological tracking data 932 using one or more of the techniques discussed herein or as otherwise may be appropriate. For example, the physiological tracking instruction set 930 may acquire physiological data 914 (e.g., neurological signal information 917 (e.g., EEG data)) from the user 110 viewing the content 902 via the device 105. Additionally, or alternatively, a user 110 may be wearing one or more sensors (e.g., such as an EEG electrode, an EDA sensor, heart rate sensor, etc.) that generates sensor data (e.g. IMU or pose data, EEG data, EDA data, heart rate data, and the like) as additional other physiological data 926. Thus, as the content 902 is presented to the user as content presentation instant 912, the physiological data 914 (e.g., neurological signal information 917, eye gaze characteristic data 915, pupillary tracking information 916, and the like) and/or other sensor data is sent to the physiological tracking instruction set 930 to track a user's physiological attributes as physiological tracking data 932, using one or more of the techniques discussed herein or as otherwise may be appropriate.

In an example implementation, the environment 900 further includes a user interface feedback instruction set 940 that is configured with instructions executable by a processor to obtain the experience data presented to the user (e.g., content 902), obtain the physiological tracking data 932, and the interaction event data 952, and generates content feedback data 942. For example, the user interface feedback instruction set 940 receives a confirmation of an interaction event, such as a selection of an icon based on determining the user is thinking of performing an action or is actually performing an action, i.e., a pinch, while the user is viewing the presentation of the content 902.

In an example implementation, the environment 900 further includes an attention-induced physiological response instruction set 950 that is configured with instructions executable by a processor to assess the user's 110 intent to interact with the interaction element 905 (e.g., determining/predicting that the user is thinking of an act-attending to an icon) based on a physiological response (e.g., tracking pupillary data 920 or neurological data 922) using one or more of the techniques discussed herein or as otherwise may be appropriate. For example, predicting an intent of the user 110 to interact with the interaction element 905 may be based on determining that the user 110 is focused on the interaction element 905 (e.g., based on eye gaze characteristic data 915 and pupillary tracking information 916) and is wanting to “click” on the element by focusing on a particular region being associated with a particular illumination characteristic (e.g., a brighter portion) and by thinking of an act—attending to an icon. In particular, the attention-induced physiological response instruction set 950 acquires physiological tracking data 932 from the physiological tracking instruction set 930 and determines the intent of the user 110 to interact with (select) the interaction element 905 during the presentation of the content 902 while the user is watching content 902. In some implementations, the attention-induced physiological response instruction set 950 can then provide interaction event data 952 (e.g., data that signals that the user selected the interaction element 905) to the user interface feedback instruction set 940 based on the attention-induced physiological response prediction (e.g., neurological or a pupillary response attention-induced pupil diameter pattern).

In some implementations, the attention-induced physiological response instruction set 950 also acquires content data 944 from the user interface feedback instruction set 940 (e.g., scene understanding data) with the physiological tracking data 932 to determine the intent of the user 110 to interact with (select) the interaction element 905 during the presentation of the content 902. For example, the content data 944 may provide a scene analysis that can be used by the attention-induced physiological response instruction set 950 to understand what the person is looking at, where they are at, etc., and improve the determination of the intent of the user to select the interaction element 905.

In some implementations, the attention-induced physiological response instruction set 950 may initiate two learning systems to engage with the user 110 using the cognitive based user interface system. First, the attention-induced physiological response instruction set 950 may learn what the user interface may or may not detect and recommend changes to the content and/or the content element 905 to be updated via the content enhancement data 954 (e.g., update the illumination characteristics of the selectable icon). Second, the attention-induced physiological response instruction set 950 may enhance a pupillary signal and/or a neural signal based on the acquired physiological tracking data 932 and content data 944 and provide physiological tracking enhancement data 956 to the physiological tracking instruction set 930 (e.g., enhance the accuracy and sensitivity of a pinch signal associated with the pupillary data or the neural data specific to each user). In other words, the quality of the received physiological signals may vary between user and/or between each experience, and the attention-induced physiological response instruction set 950 may customize each session for each user via the physiological tracking enhancement data 956 (e.g., biofeedback training for each user). The customization may be based on fine tuning a neural model for enhanced accuracy based on the pupillary and/or brain activity and user interface feedback in order to learn what the cognitive based system can detect and how to customize the enhanced physiological signals. In some implementations, the attention-induced physiological response instruction set 950 is trained and updated while the user 110 is interacting with the system, and the attention-induced physiological response instruction set 950 is then improved with the user 110 over time (e.g., a biofeedback learning system).

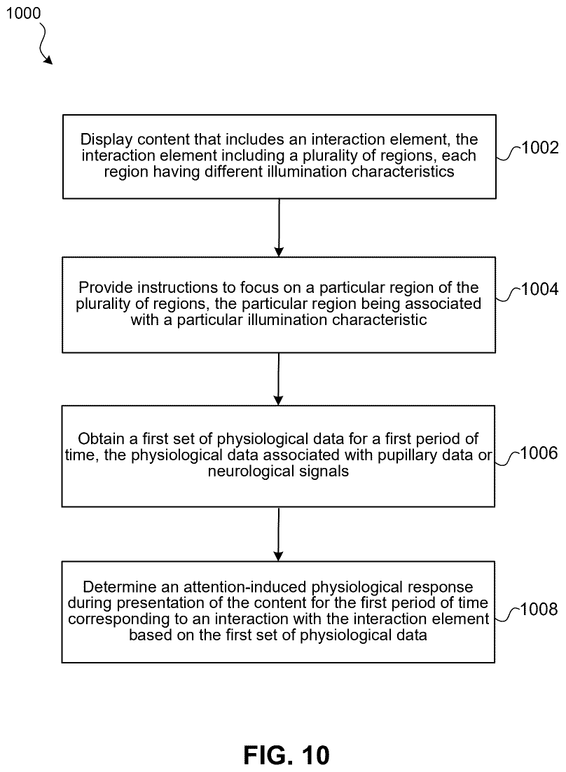

FIG. 10 is a flowchart illustrating an exemplary method 1000. In some implementations, a device such as device 105 (FIG. 1) performs the techniques of method 1000 to determine an attention-induced physiological response corresponding to an interaction with an interaction element. For example, the method 1000 may identify that, during a particular segment of an experience, the gaze characteristics (e.g., pupillary data, such as, inter alia, pupil dilation vs. constriction, stable gaze direction, velocity of pupil movements, and the like) corresponds to a user focusing on (looking at) a particular icon or user interface element (referred to herein as an “interactive element”) and based on pupillary data a prediction can be assessed whether a user is attending upon the interaction element. For example, a user may direct his or her attention to an icon (or a particular feature/attribute of an icon, such as a bright feature) in order to initiate a “click” or other interaction. This prediction of an intended interaction based on pupillary data (without using a physical movement of a body part) may be used as a user interface selection tool, device wake-up signal, etc., and might be combined with other eye or touch-based mechanisms to improve SNR, robustness, and response time. In other words, someone who may be unable to move his or her arms/hands (e.g., a person who is a quadriplegic) can look at an icon and interact with a user interface.

In some implementations, the techniques of method 1000 are performed on a mobile device, desktop, laptop, HMD, or server device. In some implementations, the method 1000 is performed on processing logic, including hardware, firmware, software, or a combination thereof. In some implementations, the method 1000 is performed on a processor executing code stored in a non-transitory computer-readable medium (e.g., a memory).

At block 1002, the method 1000 displays content that includes an interaction element, the interaction element including a plurality of regions, each region having different illumination characteristics. The content may be displayed on a graphical user interface (GUI), such as the UI illustrated in a FIGS. 6A and 6B. The illumination characteristics may include one or more icons that include stimulus features (e.g., as illustrated in FIG. 3A-3D and FIG. 4, the illumination features may include relatively dark or bright regions, motion, have higher spatial frequency content, and/or implied depth). In some implementations, the interaction element may include a selectable user interface element (e.g., an icon). In some implementations, the interaction element may include different illumination characteristics that may include one or more dark regions and one or more bright regions.

At block 1004, the method 1000 provides instructions to focus on a particular region of the plurality of regions, the particular region being associated with a particular illumination characteristic. For example, as illustrated in FIG. 4A, instructions may be provided to the user to focus on a particular region, such as, inter alia, “image white dots getting brighter”, “look at the button icon when it pos out then imaging pushing it back with your gaze”, and the like.

At block 1006, the method 1000 obtains a first set of physiological data for a first period of time, the physiological data associated with pupillary data. For example, the physiological data may be obtained from one or more sensors while a user is focused on the particular illumination characteristic. The physiological data associated with pupil dilation may include eye gaze characteristic data which may involve obtaining images of the eye or electrooculography (EOG) data, microsaccades, and/or head movements, from which pupil response/gaze direction/movement may be determined. Example pupillary response characteristics may include measuring a variability of the pupillary radius, variability may be measured based on range, variance, and/or standard deviation.

In some implementations, obtaining physiological data includes EEG amplitude/frequency, pupil modulation, eye gaze saccades, head movements, body movements (e.g., hand and/or arm movements), and the like, from which neurological response, pupil response, gaze direction/movement, and the like, may be determined. In some implementations, obtaining physiological data (e.g., neurological data, pupillary data, etc.) is associated with a gaze of a user that may involve obtaining images of the eye or electrooculography signal (EOG) data from which gaze direction and/or movement can be determined. In some implementations, the physiological data includes at least one of skin temperature, respiration, photoplethysmogram (PPG), electrodermal activity (EDA), eye gaze tracking, and pupillary movement that is associated with the user. In some implementations, obtaining physiological data includes body movements and/or head movements (e.g., obtained from an IMU or from image sensor data).

Some implementations obtain physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user will have the ability to access and otherwise find out anything that the system has collected or determined about him or her. User data is stored securely on the user's device. User data that is used as input to a machine learning model is stored securely on the user's device, for example, to ensure the user's privacy. The user's device may have a secure storage area, e.g., a secure enclave, for securing certain user information, e.g., data from image and other sensors that is used for face identification, face identification, or biometric identification. The user data associated with the user's body and/or attentive state may be stored in such a secure enclave, restricting access to the user data and restricting transmission of the user data to other devices to ensure that user data is kept securely on the user's device. User data may be prohibited from leaving the user's device and may be used only in machine learning models and other processes on the user's device.

At block 1008, the method 1000 determines an attention-induced physiological response during presentation of the content for the first period of time corresponding to an interaction with the interaction element based on the first set of physiological data. For example, initiating an action based on determining the user is imagining/thinking of a type of act (e.g., focus on attending to an icon to click it) associated with that type of interaction by focusing on the particular illumination characteristic of an icon. In other words, a physiological response may correspond to the user directing attention to a region of the regions of the interaction element based on the different illumination characteristics of the regions, and a pupil constriction may be detected when attending to a bright stimulus feature (e.g., an illumination characteristic) of a particular icon. In some implementations, the method 1000 further includes modifying the content via the user interface in response to determining the attention-induced physiological response corresponding to the interaction with the interaction element (e.g., user “clicks” on the user interface element based on user attention upon the icon).

In some implementations, customized settings may be used by the user interface based on a particular user's physiological data. In some implementations, the method 1000 further includes, updating a pupillary sensitivity level of the user interface based on the attention-induced physiological response. In some implementations, the method 1000 further includes obtaining a second set of physiological data for a second period of time, the physiological data associated with pupillary data, determining, based on the second set of physiological data, a second attention-induced physiological response during presentation of the content for the second period of time corresponding to an interaction with the interaction element, and updating the pupillary sensitivity level of the user interface based on the second attention-induced physiological response. For example, as illustrated in FIGS. 8A and 8B, the attention algorithm may be used to determine an adaptive threshold that changes over time based on the user's pupillary response data.

In some implementations, the process configured to detect user interactions of the particular type includes predicting that the user is thinking of performing an act (e.g., visual attention selection) based on recognizing (e.g., via ML model or algorithm) that a pattern exhibited in the pupillary data of the physiological data is indicative of the user thinking of performing the act (e.g., attended to the interactive element). In some implementations, there may be machine learned feedback data, a learning process in the background, and/or a guided training process to learn how “think” or “imagine” of performing the act. In some implementations, a cognitive-based user interface can continuously learn in a closed feedback loop process to improve and enhance signals and customize the feedback loop per each user (e.g., each individual person may exhibit stronger or weaker indicators of a particular intentions to select an icon via pupillary data) and/or customize to each particular experience (e.g., weaker signal detection based on, inter alia, placement/position of sensors, skin/hair conditions of the user, sensor degradation, and the like).

In some implementations, determining the attention-induced physiological response is based on a combination of neurological signals corresponding to a neurological response and pupillary data corresponding to a pupillary response.

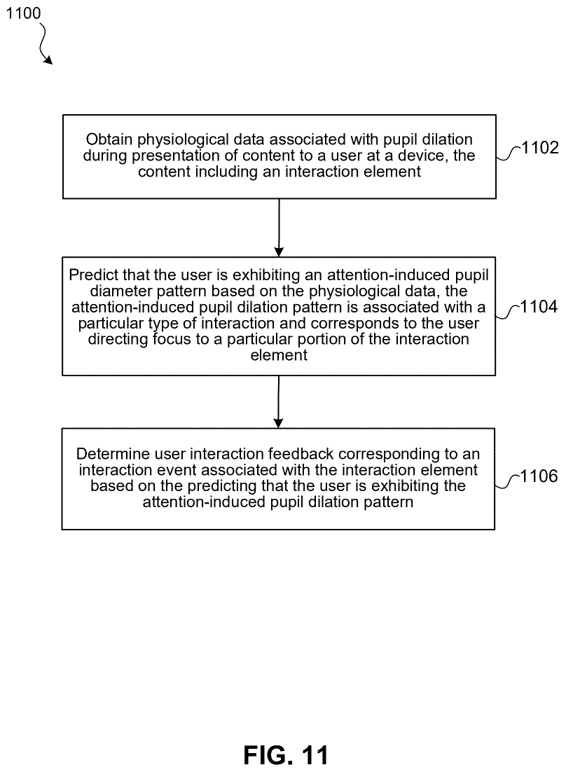

FIG. 11 is a flowchart illustrating an exemplary method 1100. In some implementations, a device such as device 105 (FIG. 1) performs the techniques of method 1100 to determine user interaction feedback corresponding to an interaction event based on predicting that the user is exhibiting an attention-induced pupil dilation pattern in accordance with some implementations. For example, the method 1100 may identify that, during a particular segment of an experience, the gaze characteristics (e.g., pupillary data, such as, inter alia, pupil dilation vs. constriction, stable gaze direction, velocity of pupil movements, and the like) corresponds to a user focusing on (looking at) a particular icon or user interface element (referred to herein as an “interactive element”) and based on the pupillary data or neurological signals (e.g., EEG data) a prediction can be assessed whether a user is thinking of an act that is associated with a particular type of interaction (e.g., a selection of an icon) based on the user exhibiting the attention-induced pupil dilation pattern. For example, a user may direct his or her attention to an icon (or a particular feature/attribute of an icon, such as a bright feature) in order to initiate a “click” or other interaction. This prediction of an intended interaction based on pupil dilation pattern (without using a physical movement of a body part) may be used as a user interface selection tool, device wake-up signal, etc., and might be combined with other eye or touch-based mechanisms to improve SNR, robustness, and response time. In other words, someone who may be unable to move his or her arms/hands (e.g., a person who is a quadriplegic) can look at an icon, perform the pupil dilation pattern with eye movements, and interact with a user interface.

In some implementations, the techniques of method 1100 are performed on a mobile device, desktop, laptop, HMD, or server device. In some implementations, the method 1100 is performed on processing logic, including hardware, firmware, software, or a combination thereof. In some implementations, the method 1100 is performed on a processor executing code stored in a non-transitory computer-readable medium (e.g., a memory).

At block 1102, the method 1100 obtains physiological data associated with pupil dilation during presentation of content to a user at the device, the content including an interaction element. For example, the physiological data associated with pupil dilation may include eye gaze characteristic data which may involve obtaining images of the eye or electrooculography (EOG) data, microsaccades, and/or head movements, from which pupil response/gaze direction/movement may be determined. The interaction element may include a selectable user interface element (e.g., an icon). In some implementations, the interaction element may include different illumination characteristics that may include one or more dark regions and one or more bright regions.

In some implementations, obtaining physiological data includes EEG amplitude/frequency, pupil modulation, eye gaze saccades, head movements, body movements (e.g., hand and/or arm movements), and the like, from which neurological response, pupil response, gaze direction/movement, and the like, may be determined. In some implementations, obtaining physiological data (e.g., pupillary data, etc.) is associated with a gaze of a user that may involve obtaining images of the eye or electrooculography signal (EOG) data from which gaze direction and/or movement can be determined. In some implementations, the physiological data includes at least one of skin temperature, respiration, photoplethysmogram (PPG), electrodermal activity (EDA), eye gaze tracking, and pupillary movement that is associated with the user. In some implementations, obtaining physiological data includes body movements and/or head movements (e.g., obtained from an IMU or from image sensor data).

Some implementations obtain physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user will have the ability to access and otherwise find out anything that the system has collected or determined about him or her. User data is stored securely on the user's device. User data that is used as input to a machine learning model is stored securely on the user's device, for example, to ensure the user's privacy. The user's device may have a secure storage area, e.g., a secure enclave, for securing certain user information, e.g., data from image and other sensors that is used for face identification, face identification, or biometric identification. The user data associated with the user's body and/or attentive state may be stored in such a secure enclave, restricting access to the user data and restricting transmission of the user data to other devices to ensure that user data is kept securely on the user's device. User data may be prohibited from leaving the user's device and may be used only in machine learning models and other processes on the user's device.

At block 1104, the method 1100 predicts that the user is exhibiting an attention-induced pupil diameter pattern based on the physiological data, the attention-induced pupil dilation pattern is associated with a particular type of interaction and corresponds to the user directing focus to a particular portion of the interaction element, such that the particular portion of the interaction element includes different illumination characteristics (e.g., characteristics associated with luminance/brightness, motion, spatial frequency, depth, etc.) than other portions of the interaction element. For example, the method 1100 may predict a particular type of interaction (e.g., a focus-on-bright-area-based selection) of an icon based on pupillary data, or other physiological data (e.g., neurological signals). In other words, detecting a click intention only using eye/pupil actions, and not requiring an input device or hand interactions (e.g., pinch). This may be based on monitoring pupil constriction and/or vergence.

In some implementations, the predicting is based on interpreting that the attention-induced pupil dilation pattern using a process (e.g., via a machine learning model or another algorithm) configured to identify pupil dilation patterns associated with focusing on user interface elements that exhibit different characteristics that is indicative of the user thinking of an act—attending to an icon. The different characteristics of the user interface elements may include characteristics associated with luminance/brightness (e.g., illumination characteristics), motion, spatial frequency, depth, and the like.

At block 1106, the method 1100 determines user interaction feedback corresponding to an interaction event associated with the interaction element based on the predicting that the user is exhibiting the attention-induced pupil dilation pattern. For example, initiating an action based on determining the user is exhibiting an attention-induced pupil diameter pattern (e.g., a focus-on-bright-area-based selection) to initiate an interaction event (click).

In various implementations, predicting that the user is thinking of performing an act may be based on determining a physiological response corresponding to the user directing attention to a region of the interaction element based on different illumination characteristics of the interaction element (e.g., pupillary response characteristics such as pupil constriction when attending to a bright feature of an icon), and then thinking of performing an act while directing attention to the interaction element. For example, pupillary response characteristics may include measuring a variability of the pupillary radius. In some implementations, variability may be measured based on range, variance, and/or standard deviation.

The following implementations may be utilized by method 1000 of FIG. 10 and/or method 1100 of FIG. 11 and are not meant to be limiting to one methodology or the other.

In some implementations, the method 1000 and/or method 1100 further includes determining user interaction feedback data corresponding to the interaction with the interaction element based on the attention-induced physiological response. For example, identify customized settings for the user based on his or her physiological data. The customized settings may be based on feedback data (e.g., machine learning), a learning process in the background, guided training process, and the like. Thus, a cognitive UI can then continuously learn in a closed feedback loop process to learn what cognitive UI may be able to detect and can enhance the attention-induced physiological response corresponding to an interaction with the interaction element. In some implementations, the user interaction feedback data includes providing instructions to the user via the user interface to initiate the attention-induced physiological response corresponding to an interaction event with the interaction element while thinking of thinking of an act—attending to the element to select the element. For example, guide/train a user to initiate an interaction event (click). In other words, train a user to attend to stimulate a pupillary signal to attention-induced physiological response to a particular illumination characteristic of an icon and/or focus on initiating a pinch (e.g., “focus on imagining your hand moving to expand an orb”) in order to replace pinch and enable hands free use.

In some implementations, the method 1000 and/or method 1100 further includes, in response to determining, based on the sensor data, that the act associated with the particular type of interaction is performed, obtaining additional physiological data associated with neurological signals, and updating a prediction model associated with the user performing the act associated with the particular type of interaction based on the additional physiological data. For example, an act is performed, and the pupillary data confirms the user intentions to perform a “click” and allows for quicker response in a subsequent act (e.g., the cognitive-based user interface improves on the accuracy of detecting and predicting an intent action).

In various implementations, predicting that the user is thinking of performing an act (e.g., attending to the icon) may be based on determining a physiological response corresponding to the user directing attention to a region of the interaction element based on different illumination characteristics of the interaction element (e.g., pupillary response characteristics such as pupil constriction when attending to a bright feature of an icon), and then thinking of performing an act while directing attention to the interaction element. For example, pupillary response characteristics may include measuring a variability of the pupillary radius. In some implementations, variability may be measured based on range, variance, and/or standard deviation.

In some implementations, the method 1000 and/or the method 1100 further includes, obtaining additional physiological data associated with body movements (e.g., body tracking data), and updating a prediction model associated with predicting that the user is thinking of performing the act associated with the particular type of interaction based on the additional physiological data (e.g., a user's body movements may be tracked with cameras and may also be used for prediction). For example, a body tracking algorithm may track arm motion to reduce false positives for identifying a false pinch or another type of movement. For example, arm motion may trigger neural signals that are similar to a pinch. The device 105 may include external facing cameras to capture images (e.g., RGB and/or depth data) of the user's body, and that image data may be used by a body tracking algorithm that can identify periods of rapid arm motion. Thus, a attention-induced physiological response prediction system can separate out arm motion neural signals to decrease potential false positives for a pinch signal.

In some implementations, determining the neurological response during the presentation of the interaction element is based on determining a variability of the neurological response to a threshold. An example threshold limit for the variability of the neurological response may be based on a machine learning model output. For example, if the machine learning model takes the physiological data as input and outputs a probability of click intent (e.g., 70%), then a determination may be made that any neurological response causing a probability under 70% is no click, while a neurological response leading to a machine learning model output at or above 70% is a click. The same thresholding may be applied to a pupillary response.

Another type of threshold could be an outlier detection, e.g., if the neurological response, pupillary response, or other physiological data changes beyond an accepted range, and that data may be rejected and considered as noise. Likewise, if response changes are so small that the system would have low confidence in measuring such a small change, the system might also reject that data as noise.

In some implementations, the user interaction feedback corresponding to an interaction event associated with the interaction element is classified using a machine learning technique. In some implementations, the interaction with the interaction element is classified using a machine learning technique based on the attention-induced physiological response and the different illumination characteristics of the regions. In some implementations, a machine learning algorithm may be determined based predicting a “click” or “no click” for each time point based on the presence of a prediction of an attention-induced physiological response (e.g., assessed that a user is thinking of performing an act that is associated with a particular type of interaction, e.g., a selection). In some implementations, determining an interaction event includes determining scene-induced pupil response variation characteristics for the interaction element, and determining the interaction event during the presentation of the interaction element based on the scene-induced pupil response variation characteristics for the regions of the interaction element. The pupillary response may be a direction of the pupillary response, a velocity of the pupillary response, or pupillary fixations (e.g., derived from eye gaze dynamics and saccade characteristics). For example, when a user's gaze intersects with a user interface element (e.g., interaction element 415 of FIG. 4A), and there is a determination of a prediction of an act (e.g., a selection of an icon based on user attention), a machine learning protocol can predict a “click” or “no click” for each time point, based on the presence of a prediction of an attention-induced physiological response. Therefore, in some implementations, the interaction event may be classified using a machine learning technique based on the pupil response and/or the neurological response (e.g., a machine learning “click” model).

In some implementations, the method 1000 and/or the method 1100 further includes modifying the content via the user interface in response to determining the attention-induced physiological response corresponding to the interaction with the interaction element. For example, if the system determines that the user “clicks” on the UI element, then an action may be performed.

In some implementations, the machine learning model is a neural network (e.g., an artificial neural network), decision tree, support vector machine, Bayesian network, or the like. These labels may be collected from the user beforehand, or from a population of people beforehand, and fine-tuned later on individual users. Creating this labeled data may require many users going through an experience (e.g., a meditation experience) where the users listen to natural sounds with intermixed natural-probes (e.g., an auditory stimulus) and then randomly are asked how focused or relaxed they were (e.g., interaction event) shortly after a probe was presented. The answers to these questions can generate a label for the time prior to the question and a deep neural network or deep long short term memory (LSTM) network might learn a combination of features specific to that user or task given those labels (e.g., low interaction event, high interaction event, etc.).

In some implementations, the method 1000 and/or the method 1100 further includes adjusting content in response to determining the interaction event (e.g., attending to an icon for a selection based on pupillary data). For example, as illustrated in the system flow diagram of environment 900 of FIG. 9, when is determined that user's intent is to “click” (e.g., interact/focus) on the interaction element 905, the attention-induced physiological response instruction set 950 provides interaction event data 952 to the content instruction set 910 to update the content (e.g., change the content based on the selection of the icon-interaction element 905).

In some implementations, the techniques described herein obtain physiological data (e.g., pupillary data, EEG amplitude/frequency data, pupil modulation, eye gaze saccades, head movements, etc.) from the user based on identifying typical interactions of the user with the experience. For example, the techniques may determine that a variability of brain activity and eye gaze characteristic of the user correlates with an interaction with the experience. Additionally, the techniques described herein may then adjust a visual characteristic of the experience, or adjust/change a sound associated with the interaction element, to enhance physiological response data associated with future interactions with the experience and/or the interaction element presented within the experience. Moreover, in some implementations, changing an interaction element after the user interacts with, or is predicted to interact with, the experience informs the physiological response of the user in subsequent interactions with the interaction element or a particular segment of the experience. For example, the user may present an anticipatory physiological response associated with the change within the interaction element (e.g., a change in illuminance of the interaction element). Thus, in some implementations, the technique identifies a prediction of an intent of the user to perform an act to interact (attend) with the interaction element based on an anticipatory physiological response. For example, the technique may adapt or train an instruction set by capturing or storing physiological data of the user based on the interaction of the user with the experience, and may detect a future intention of the user to interact with the experience by identifying a physiological response of the user in anticipation of the presentation of the enhanced/updated interaction element.

In some implementations, there may be machine learned feedback data, a learning process in the background, and/or a guided training process to learn how “think” or “imagine” of performing the act. In some implementations, a cognitive-based user interface can continuously learn in a closed feedback loop process to improve and enhance the pupillary data signals and customize the feedback loop per each user (e.g., each individual person may exhibit stronger or weaker indicators of a particular thought or attention towards an icon) and/or customize to each particular experience (e.g., weaker signal detection based on, inter alia, placement/position of sensors, skin/hair conditions of the user, sensor degradation, and the like).

In some implementations, customization of the experience could be controlled by the user. For example, a user could select the experience he or she desires, such as he or she can choose the ambience, background scene, music, etc. Additionally, the user could alter the threshold of selecting the interactive element. For example, the user can customize the sensitivity of triggering the interactive element based on prior experience of a session. For example, a user may desire to not have as many notifications and allow some mind wandering (e.g., eye position deviations) or some body/arm movements before an interactive element is triggered. Thus, particular experiences can be customized on triggering a threshold when higher criteria is met. For example, a user may have to look at a particular interactive element for longer (or shorter) to toggle the interactive element (e.g. shakes or flashes) and/or a longer (or shorter) threshold (e.g., two or more seconds) after the toggle to actually select the interactive element.

In some embodiments, the method 1000 or method 1100 determines a context of the experience based on sensor data of the environment. For example, determining a context may involve using computer vision to generate a scene understanding of the visual and/or auditory attributes of the environment—where is the user, what is the user doing, what objects are nearby. Additionally, a scene understanding of the content presented to the user could be generated that includes the visual and/or auditory attributes of what the user was watching.

In some embodiments, different contexts of the content presented and the environment are analyzed to determine where the user is, what the user is doing, what objects or people are nearby in the environment or within the content, what the user did earlier (e.g., meditated in the morning). Additionally, context analysis may include image analysis (semantic segmentation), audio analysis (jarring sounds), location sensors (where user is), motion sensors (fast moving vehicle), and even access other user data (e.g., a user's calendar). In an exemplary implementation, the method 1000 or method 1100 may further include determining the context of the experience by generating a scene understanding of the environment based on the sensor data of the environment, the scene understanding including visual or auditory attributes of the environment, and determining the context of the experience based on the scene understanding of the environment.

In some implementations, the sensor data includes image data, and generating the scene understanding is based at least on performing semantic segmentation of the image data and detecting one or more objects within the environment based on the semantic segmentation. In some implementations, determining the context of the experience includes determining an activity of the user based on the scene understanding of the environment. In some implementations, the sensor data includes location data of the user, and determining the context of the experience includes determining a location of the user within the environment based on the location data.

In some implementations, determining the context of the experience includes determining an activity of the user based on a user's schedule. For example, the system may access a user's calendar to determine if a particular event is occurring when the particular interaction event is assessed. For example, different applications may include different interaction elements to be provided to the user to select via his or her neurological data and/or pupillary response (eye gaze characteristics).

In some implementations, one or more pupillary or EEG characteristics may be determined, aggregated, and used to classify the prediction of the user's intent to perform an act to determine an interaction event occurrence using statistical or machine learning techniques. In some implementations, the physiological data is classified based on comparing the variability of the physiological data to a threshold.

In some implementations, the method 1000 and/or the method 1100 further includes adjusting content corresponding to the experience based on the interaction event (e.g., customized to the interaction event). For example, content recommendation for a content developer can be provided based on determining interaction events during the presented experience and changes of the experience or content presented therein. For example, the user may focus well (e.g., imagine or think about performing the pinch better) when particular types of content are provided. In some implementations, the method 1000 and/or method 1100 may further include identifying content based on similarity of the content to the experience, and providing a recommendation of the content to the user based on determining that the user has the interaction event during the experience (e.g., mind wandering). In some implementations, the method 1000 and/or the method 1100 may further include customizing content included in the experience based on the interaction event (e.g., breaking the content into smaller pieces).

In some implementations, an estimator or statistical learning method is used to better understand or make predictions about the physiological data (e.g., pupillary data characteristics, head movements, etc.). For example, statistics for pupillary data may be estimated by sampling a dataset with replacement data (e.g., a bootstrap method).

In some implementations, the techniques could be trained on many sets of user physiological data and then adapted to each user individually. For example, content creators can customize an experience (e.g., an instructional video) based on the user physiological data, such as a user may require background music, different ambient lighting for learning, or require more or less audio or visual cues to continue to maintain an attention state that is better suited for predicting an attention-induced physiological response of the user.

In some implementations, the techniques described herein can account for real-world environment 100 of the user 110 (e.g., visual qualities such as luminance, contrast, semantic context) in its evaluation of how much to modulate or adjust the presented content or interactive elements to enhance the physiological response (e.g., pupillary response) of the user 110 to a visual characteristic (e.g., interactive elements).

In some implementations, the techniques described herein can utilize a training or calibration sequence to adapt to the specific physiological characteristics of a particular user 110, as described herein with reference to FIG. 6. In some implementations, the techniques present the user 110 with a training scenario in which the user 110 is instructed to interact with on-screen items (e.g., interactive objects). By providing the user 110 with a known intent or area of interest (e.g., via instructions), the techniques can record the user's physiological data (e.g., neurological data, pupillary data, body/head movement data, etc.) and identify a pattern associated with the user's physiological data. In some implementations, the techniques can change a visual characteristic (e.g., a feedback mechanism) associated with content in order to further adapt to the unique physiological characteristics of the user 110. For example, the techniques can direct a user to mentally select a button (e.g., an interactive element) associated with an identified area in the center of the screen on the count of three and record the user's physiological data (e.g., neurological data, pupillary data, body/head movement data, etc.) to identify a pattern associated with the user's interaction event. Moreover, the techniques can change or alter a visual characteristic associated with the feedback mechanism in order to identify a pattern associated with the user's physiological response to the altered visual characteristic. In some implementations, the pattern associated with the physiological response of the user 110 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying).

In some implementations, a machine learning model (e.g., a trained neural network) is applied to identify patterns in physiological data (e.g., neurological signals), including identification of physiological responses to presentation of content during a particular experience (e.g., education, meditation, instructional, etc.). Moreover, the machine learning model may be used to match the patterns with learned patterns corresponding to indications of interest or intent of the user 110 to interact with the interaction element. In some implementations, the techniques described herein may learn patterns specific to the particular user 110. For example, the techniques may learn from determining that a peak pattern represents an indication of interest or intent of the user 110 in response to a particular visual characteristic within the content and use this information to subsequently identify a similar peak pattern as another indication of interest or intent of the user 110. Such learning can take into account the user's relative interactions with multiple visual characteristics, in order to further adjust the visual characteristic and enhance the user's physiological response to the experience and the presented content (e.g., focusing on particular areas of content versus other distracting areas).

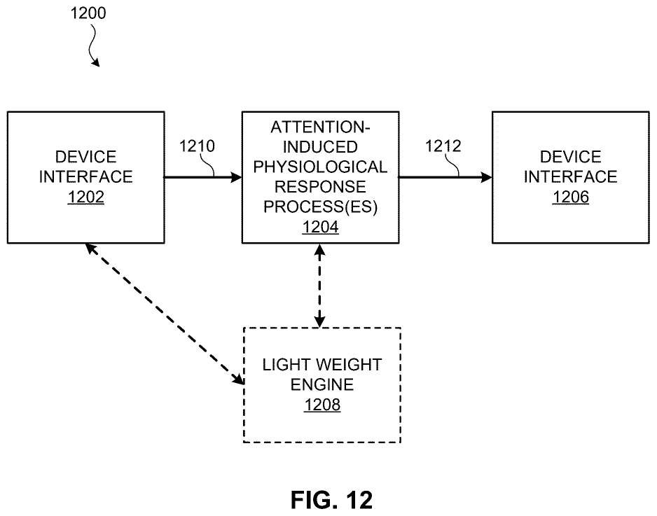

FIG. 12 illustrates a system diagram that includes a user device 1200 (e.g., device 105, device 125, etc.) for an attention-induced physiological response process including a device interface 1202, attention-induced physiological response process(es) 1204, and a device interface 1206.

Inputs 1210 from device interface 1202 can be fed to attention-induced physiological response process(es) 1204. attention-induced physiological response process(es) 1204 can include one or more learning-based and/or non-learning-based models for perceiving, synthesizing, and inferring information. Persons skilled in the art will appreciate that the attention-induced physiological response process(es) 1204 can include any suitable number of attention-induced physiological response processes to generate output 1212 based on input 1210. In some embodiments, user device 1200 may optionally elect to use light weight engine instead of attention-induced physiological response process(es) 1204 to process data in situations where only one modality of data is being processed. The light weight engine 1208 may be a non-learning network. Output from attention-induced physiological response process(es) 1204 is provided to device interface 1206, where interface 1206 disambiguates the recommendations. Based on current context and default settings provided by a user, interface 1206 may select a subset of recommendations to surface to the user.

Persons of ordinary skill in the art will appreciate that attention-induced physiological response process(es) 1204 can include any suitable machine learning models that are well-known or widely available such as regression techniques, classification techniques, neural networks, and deep learning networks. For instance, attention-induced physiological response process(es) 1204 can include neural networks such as [remove ones that don't apply] Artificial Neural Network (ANN), Convolutional Neural Network (CNN), Recurrent Neural Network (RNN), Adversarial Network (GAN), Reinforcement Learning Model (RLM), Encoder/Decoder Networks, and/or Transformer-Based Models (e.g., Bidirectional Encoder Representations from Transformers (BERT), Generative Pre-trained Transformer (GPT), and/or a multi-modal large language model (LLM)). Additionally or alternatively, persons of ordinary skill in the art will appreciate that attention-induced physiological response process(es) 1204 can be any suitable non-learning processes such as rule-based systems, heuristics, decision trees, knowledge-based systems, statistical or stochastic systems, and expert systems.

In instances where attention-induced physiological response process(es) 1204 is a machine-learning based model, attention-induced physiological response process(es) 1204 can be trained to identify one or more conditions associated with performance by a user of a plurality of tasks using one or more well-known or widely available training techniques such as supervised learning, semi-supervised learning, unsupervised learning, and/or reinforcement learning techniques. The training data can include historical task lists and associated performances and/or conditions associated with completing one or more tasks.

In some embodiments, attention-induced physiological response process(es) 1204 can be deployed as one or more generative models, where content is automatically generated by one or more computers in response to a request to generate the content. The automatically-generated content is optionally generated on-device (e.g., generated at least in part by a computer system at which a request to generate the content is received) and/or generated off-device (e.g., generated at least in part by one or more nearby computers that are available via a local network or one or more computers that are available via the internet). This automatically-generated content optionally includes visual content (e.g., images, graphics, and/or video), audio content, and/or text content.

In some embodiments, novel automatically-generated content is referred to as generative content (e.g., generative images, generative graphics, generative video, generative audio, and/or generative text). Generative content is typically generated based on a prompt input 1210 to the attention-induced physiological response process(es) 1204. Device interface 1202 optionally includes one or more pre-processing steps to adjust the input before it is used by an AI model to generate an output (e.g., adjustment to a user-provided prompt, creation of a system-generated prompt, and/or AI model selection). Device interface 1206 optionally includes one or more post-processing steps to adjust the output 1212 by the attention-induced physiological response process(es) 1204 (e.g., passing the AI model output to a different AI model, upscaling, downscaling, cropping, formatting, and/or adding or removing metadata) before the output 1212 of the attention-induced physiological response process(es) 1204 used for other purposes such as being provided to a different software process for further processing or being presented (e.g., visually or audibly) to a user.

A prompt input 1210 for generating generative content can include one or more of: one or more words (e.g., a natural language prompt that is written or spoken), one or more images, one or more drawings, and/or one or more videos. Generative pre-trained transformer models are a type of LLM that can be effective at generating novel generative content based on a prompt input 1210. In some embodiments, the attention-induced physiological response process(es) 1204 uses a prompt input 1210 that includes text to generate either different generative text, generative audio content, and/or generative visual content. In other embodiments, the attention-induced physiological response process(es) 1204 uses a prompt input 1210 that includes visual content and/or an audio content to generate generative text (e.g., a transcription of audio and/or a description of the visual content). In yet other embodiments, the attention-induced physiological response process(es) 1204 uses a prompt input 1210 that includes multiple types of content (e.g., text, images, audio, video, and/or other sensor data) to generate generative content. A prompt input 1210 sometimes also includes values for one or more parameters indicating an importance of various parts of the prompt. Some prompt inputs 1210 include a structured set of instructions that can be provided to the attention-induced physiological response process(es) 1204 that include phrasing, a specified style, relevant context (e.g., starting point content and/or one or more examples), and/or a role for the attention-induced physiological response process(es) 1204.

Generative content is generally based on the prompt but is not deterministically selected from pre-generated content and is, instead, generated using the prompt as a starting point. In some embodiments, pre-existing content (e.g., audio, text, and/or visual content) is used as part of the prompt for creating generative content (e.g., the pre-existing content is used as a starting point for creating the generative content). For example, a prompt input 1210 could request that a block of text be summarized or rewritten in a different tone, and the output 1212 would be generative text that is summarized or written in the different tone. Similarly a prompt input 1210 could request that visual content be modified to include or exclude content specified by a prompt (e.g., removing an identified feature in the visual content, adding a feature to the visual content that is described in a prompt, changing a visual style of the visual content, and/or creating additional visual elements outside of a spatial or temporal boundary of the visual content that are based on the visual content). In some embodiments, a random or pseudo-random seed is used as part of the prompt input 1210 for creating generative content (e.g., the random or pseudo-random seed content is used as a starting point for creating the generative content). For example, when generating an image from a diffusion model, a random noise pattern is iteratively denoised based on the prompt input 1210 to generate an image. While specific types of attention-induced physiological response processes 1204 have been described herein, it should be understood that a variety of different attention-induced physiological response processes could be used to generate generative content based on a prompt.

In instances where attention-induced physiological response process(es) 1204 is a non-learning-based system, attention-induced physiological response process(es) 1204 can use a pre-defined set of rules or a pre-defined structure to make decisions based on the inputs that the process sees. For example, the attention-induced physiological response process(es) 1204 can sequentially process each word in an input phrase and count the number of times that the word appears. It can then prepare the summary using words that appear with the greatest frequency.

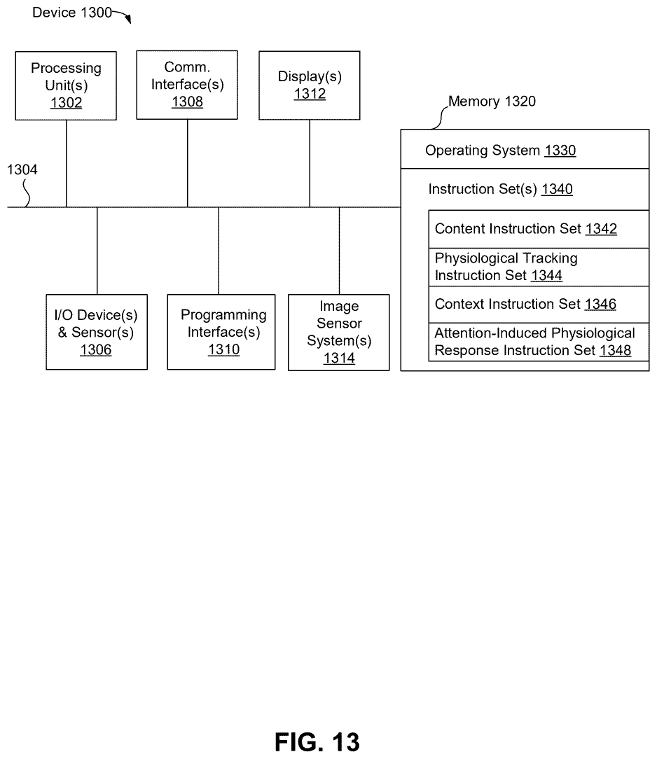

FIG. 13 is a block diagram of an example device 1300. Device 1300 illustrates an exemplary device configuration for device 10. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 105 includes one or more processing units 1302 (e.g., microprocessors, ASICs, FPGAs, GPUs, CPUs, processing cores, and/or the like), one or more input/output (I/O) devices and sensors 1306, one or more communication interfaces 1308 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 1310, one or more displays 1312, one or more interior and/or exterior facing image sensor systems 1314, a memory 1320, and one or more communication buses 1304 for interconnecting these and various other components.

In some implementations, the one or more communication buses 1304 include circuitry that interconnects and controls communications between system components. In some implementations, the one or more I/O devices and sensors 1306 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 1312 are configured to present a view of a physical environment or a graphical environment to the user. In some implementations, the one or more displays 1312 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 1312 correspond to diffractive, reflective, polarized, holographic, etc. waveguide displays. In one example, the device 105 includes a single display. In another example, the device 105 includes a display for each eye of the user.

In some implementations, the one or more image sensor systems 1314 are configured to obtain image data that corresponds to at least a portion of the physical environment 5. For example, the one or more image sensor systems 1314 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 1314 further include illumination sources that emit light, such as a flash. In various implementations, the one or more image sensor systems 1314 further include an on-camera image signal processor (ISP) configured to execute a plurality of processing operations on the image data.

The memory 1320 includes high-speed random-access memory, such as DRAM, SRAM, DDR RAM, or other random-access solid-state memory devices. In some implementations, the memory 1320 includes non-volatile memory, such as one or more magnetic disk storage devices, optical disk storage devices, flash memory devices, or other non-volatile solid-state storage devices. The memory 1320 optionally includes one or more storage devices remotely located from the one or more processing units 1302. The memory 1320 includes a non-transitory computer readable storage medium.

In some implementations, the memory 1320 or the non-transitory computer readable storage medium of the memory 1320 stores an optional operating system 1330 and one or more instruction set(s) 1340. The operating system 1330 includes procedures for handling various basic system services and for performing hardware dependent tasks. In some implementations, the instruction set(s) 1340 include executable software defined by binary information stored in the form of electrical charge. In some implementations, the instruction set(s) 1340 are software that is executable by the one or more processing units 1302 to carry out one or more of the techniques described herein.

The instruction set(s) 1340 include a content instruction set 1342, a physiological tracking instruction set 1344, a context instruction set 1346, and an attention-induced physiological response instruction set 1348. The instruction set(s) 1340 may be embodied a single software executable or multiple software executables.

In some implementations, the content instruction set 1342 is executable by the processing unit(s) 1302 to provide and/or track content for display on a device. The content instruction set 1342 may be configured to monitor and track the content over time (e.g., during an experience such as an education session) and/or to identify change events that occur within the content. In some implementations, the content instruction set 1342 may be configured to inject change events into content (e.g., feedback mechanisms) using one or more of the techniques discussed herein or as otherwise may be appropriate. To these ends, in various implementations, the instruction includes instructions and/or logic therefor, and heuristics and metadata therefor.

In some implementations, the physiological tracking instruction set 1344 is executable by the processing unit(s) 1302 to track a user's physiological attributes (e.g., EEG amplitude/frequency, pupil modulation, eye gaze saccades, body/head movement data, etc.) using one or more of the techniques discussed herein or as otherwise may be appropriate. To these ends, in various implementations, the instruction includes instructions and/or logic therefor, and heuristics and metadata therefor.

In some implementations, the context instruction set 1346 is executable by the processing unit(s) 1302 to determine a context of the experience and/or the environment (e.g., create a scene understanding to determine the objects or people in the content or in the environment, where the user is, what the user is watching, etc.) using one or more of the techniques discussed herein (e.g., object detection, facial recognition, etc.) or as otherwise may be appropriate. To these ends, in various implementations, the instruction includes instructions and/or logic therefor, and heuristics and metadata therefor.

In some implementations, the attention-induced physiological response instruction set 1348 is executable by the processing unit(s) 1302 to determine an attention-induced physiological response associated with an intent of the user to interact with an interaction element (e.g., an interaction event determination) based on physiological data associated with pupillary data and/or neurological signals (e.g., EEG data) using one or more of the techniques discussed herein or as otherwise may be appropriate. To these ends, in various implementations, the instruction includes instructions and/or logic therefor, and heuristics and metadata therefor.

Although the instruction set(s) 1340 are shown as residing on a single device, it should be understood that in other implementations, any combination of the elements may be located in separate computing devices. Moreover, FIG. 13 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

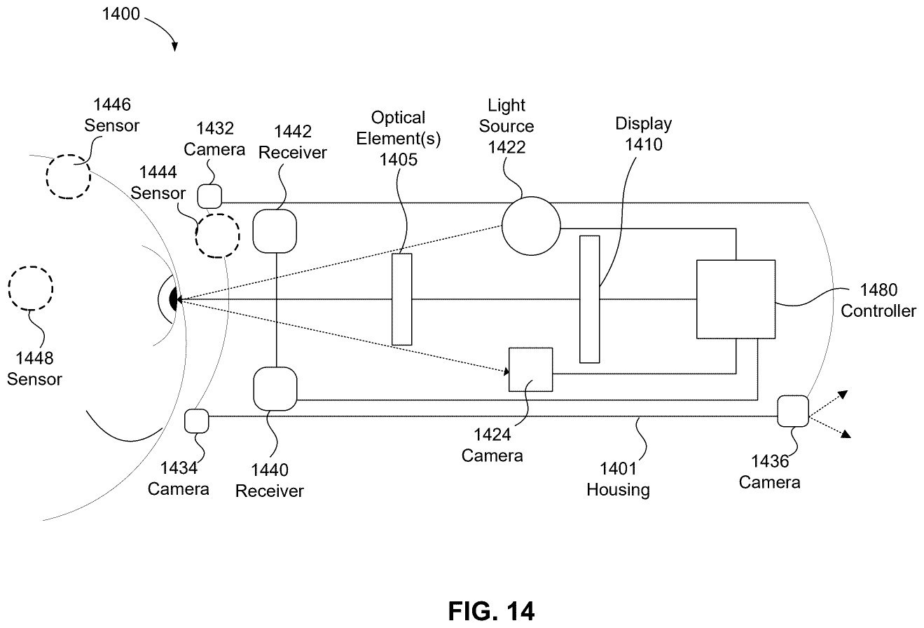

FIG. 14 illustrates a block diagram of an exemplary head-mounted device 1400 in accordance with some implementations. The head-mounted device 1400 includes a housing 1401 (or enclosure) that houses various components of the head-mounted device 1400. The housing 1401 includes (or is coupled to) an eye pad (not shown) disposed at a proximal (to the user) end of the housing 1401. In various implementations, the eye pad is a plastic or rubber piece that comfortably and snugly keeps the head-mounted device 1400 in the proper position on the face of the user 110 (e.g., surrounding the eye of the user).

The housing 1401 houses a display 1410 that displays an image, emitting light towards or onto the pupil of an eye of a user. In various implementations, the display 1410 emits the light through an eyepiece having one or more optical elements 1405 that refracts the light emitted by the display 1410, making the display appear to the user to be at a virtual distance farther than the actual distance from the eye to the display 1410. For example, optical element(s) 1405 may include one or more lenses, a waveguide, other diffraction optical elements (DOE), and the like. For the user to be able to focus on the display 1410, in various implementations, the virtual distance is at least greater than a minimum focal distance of the eye (e.g., 7 cm). Further, in order to provide a better user experience, in various implementations, the virtual distance is greater than 1 meter.

The housing 1401 also houses a tracking system including one or more light sources 1422, camera 1424, and a controller 1480. The one or more light sources 1422 emit light onto the eye of the user that reflects as a light pattern (e.g., a circle of glints) that can be detected by the camera 1424. Based on the light pattern, the controller 1480 can determine an eye tracking characteristic of the user. For example, the controller 1480 can determine a gaze direction or a blinking state (eyes open or eyes closed) of the user. As another example, the controller 1480 can determine a pupil center, a pupil size, or a point of regard associated with the pupil. Thus, in various implementations, the light is emitted by the one or more light sources 1422, reflects off the eye of the user, and is detected by the camera 1424. In various implementations, the light from the eye of the user is reflected off a hot mirror or passed through an eyepiece before reaching the camera 1424.

The display 1410 emits light in a first wavelength range and the one or more light sources 1422 emit light in a second wavelength range. Similarly, the camera 1424 detects light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm).

In various implementations, eye tracking (or, in particular, a determined gaze direction) is used to enable user interaction (e.g., the user selects an option on the display 1410 by looking at it), provide foveated rendering (e.g., present a higher resolution in an area of the display 1410 the user is looking at and a lower resolution elsewhere on the display 1410), or correct distortions (e.g., for images to be provided on the display 1410). In various implementations, the one or more light sources 1422 emit light towards the eye of the user, which reflects in the form of a plurality of glints.

In various implementations, the camera 1424 is a frame/shutter-based camera that, at a particular point in time or multiple points in time at a frame rate, generates an image of the eye of the user. Each image includes a matrix of pixel values corresponding to pixels of the image which correspond to locations of a matrix of light sensors of the camera. In implementations, each image is used to measure or track pupil dilation by measuring a change of the pixel intensities associated with one or both of a user's pupils.

In various implementations, the housing 1401 also houses a camera system for capturing images of the user and/or the external environment. As illustrated in FIG. 14, the camera system may include camera 1432, camera 1434, and camera 1436. In various implementations, the camera 1432, camera 1434, and camera 1436 are frame/shutter-based cameras that, at a particular point in time or multiple points in time at a frame rate, can generate an image of the face of the user 110 or capture an external physical environment. For example, camera 1432 captures images of the user's face below the eyes, camera 1434 captures images of the user's face above the eyes, and camera 1436 captures the external environment of the user (e.g., environment 100 of FIG. 1). The images captured by camera 1432, camera 1434, and camera 1436 may include light intensity images (e.g., RGB) or depth image data (e.g., Time-of-Flight, infrared, etc.).

In various implementations, the housing 1401 also houses a neurological sensing system (e.g., EEG monitoring) for capturing neurological signals of the user. As illustrated in FIG. 14, the neurological sensing system may include receivers 1440, 1442, and sensors 1444, 1446, and 1448. In various implementations, sensor 1444, sensor 1446, and sensor 1448 are electrodes for capturing various neurological signals that are received by the receiver 1440 and/or receiver 1442 either via wireless or direct electrical connections (wired). The receiver 1440 and/or receiver 1442 sends the neurological signals from the sensors to the controller 1480. In various implementations, the controller 1480 can determine various neural processes that are mediated through the electrical signals captured by the sensors.

In various implementations, the sensors 1444, 1446, and 1448 may be located on the outer surface of the housing 1401 (e.g., sensor 1444, which would contact the skin of the user when worn), or the sensors may be positioned on a head strap or other mounting system for the head worn device 1400 (e.g., sensors 1446, 1448, such that when the device 1400 is worn the sensors would contact the head of the user 110). The illustration of FIG. 14 is for illustrative purposes and is not meant to be limiting regarding the number or sensors and/or receivers, and the various positioning of the neurological sensing system components (e.g., location of the electrodes). For example, the electrodes of the neurological sensing system may be located on a separately worn device or head strap that could send the various neurological signals to the device 1400.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

Some embodiments described herein can include use of learning and/or non-learning-based process(es). The use can include collecting, pre-processing, encoding, labeling, organizing, analyzing, recommending and/or generating data. Entities that collect, share, and/or otherwise utilize user data should provide transparency and/or obtain user consent when collecting such data. The present disclosure recognizes that the use of the data in the can be used to benefit users. For example, the data can be used to train models that can be deployed to improve performance, accuracy, and/or functionality of applications and/or services. Accordingly, the use of the data enables the attention-induced physiological response processes to adapt and/or optimize operations to provide more personalized, efficient, and/or enhanced user experiences. Such adaptation and/or optimization can include tailoring content, recommendations, and/or interactions to individual users, as well as streamlining processes, and/or enabling more intuitive interfaces. Further beneficial uses of the data in the attention-induced physiological response processes are also contemplated by the present disclosure.

The present disclosure contemplates that, in some embodiments, data used by attention-induced physiological response processes includes publicly available data. To protect user privacy, data may be anonymized, aggregated, and/or otherwise processed to remove or to the degree possible limit any individual identification. As discussed herein, entities that collect, share, and/or otherwise utilize such data should obtain user consent prior to and/or provide transparency when collecting such data. Furthermore, the present disclosure contemplates that the entities responsible for the use of data, including, but not limited to data used in association with attention-induced physiological response processes, should attempt to comply with well-established privacy policies and/or privacy practices.

For example, such entities may implement and consistently follow policies and practices recognized as meeting or exceeding industry standards and regulatory requirements for developing and/or training attention-induced physiological response processes. In doing so, attempts should be made to ensure all intellectual property rights and privacy considerations are maintained. Training should include practices safeguarding training data, such as personal information, through sufficient protections against misuse or exploitation. Such policies and practices should cover all stages of the attention-induced physiological response processes development, training, and use, including data collection, data preparation, model training, model evaluation, model deployment, and ongoing monitoring and maintenance. Transparency and accountability should be maintained throughout. Such policies should be easily accessible by users and should be updated as the collection and/or use of data changes. User data should be collected for legitimate and reasonable uses of the entity and not shared or sold outside of those legitimate uses. Further, such collection and sharing should occur through transparency with users and/or after receiving the informed consent of the users. Additionally, such entities should consider taking any needed steps for safeguarding and securing access to such data and ensuring that others with access to the data adhere to their privacy policies and procedures. Further, such entities should subject themselves to evaluation by third parties to certify, as appropriate for transparency purposes, their adherence to widely accepted privacy policies and practices. In addition, policies and/or practices should be adapted to the particular type of data being collected and/or accessed and tailored to a specific use case and applicable laws and standards, including jurisdiction-specific considerations.

In some embodiments, attention-induced physiological response processes may utilize models that may be trained (e.g., supervised learning or unsupervised learning) using various training data, including data collected using a user device. Such use of user-collected data may be limited to operations on the user device. For example, the training of the model can be done locally on the user device so no part of the data is sent to another device. In other implementations, the training of the model can be performed using one or more other devices (e.g., server(s)) in addition to the user device but done in a privacy preserving manner, e.g., via multi-party computation as may be done cryptographically by secret sharing data or other means so that the user data is not leaked to the other devices.

In some embodiments, the trained model can be centrally stored on the user device or stored on multiple devices, e.g., as in federated learning. Such decentralized storage can similarly be done in a privacy preserving manner, e.g., via cryptographic operations where each piece of data is broken into shards such that no device alone (i.e., only collectively with another device(s)) or only the user device can reassemble or use the data. In this manner, a pattern of behavior of the user or the device may not be leaked, while taking advantage of increased computational resources of the other devices to train and execute the ML model. Accordingly, user-collected data can be protected. In some implementations, data from multiple devices can be combined in a privacy-preserving manner to train an ML model.

In some embodiments, the present disclosure contemplates that data used for attention-induced physiological response processes may be kept strictly separated from platforms where the attention-induced physiological response processes are deployed and/or used to interact with users and/or process data. In such embodiments, data used for offline training of the attention-induced physiological response processes may be maintained in secured datastores with restricted access and/or not be retained beyond the duration necessary for training purposes. In some embodiments, the attention-induced physiological response processes may utilize a local memory cache to store data temporarily during a user session. The local memory cache may be used to improve performance of the attention-induced physiological response processes. However, to protect user privacy, data stored in the local memory cache may be erased after the user session is completed. Any temporary caches of data used for online learning or inference may be promptly erased after processing. All data collection, transfer, and/or storage should use industry-standard encryption and/or secure communication.

In some embodiments, as noted above, techniques such as federated learning, differential privacy, secure hardware components, homomorphic encryption, and/or multi-party computation among other techniques may be utilized to further protect personal information data during training and/or use of the attention-induced physiological response processes. The attention-induced physiological response processes should be monitored for changes in underlying data distribution such as concept drift or data skew that can degrade performance of the attention-induced physiological response processes over time.

In some embodiments, the attention-induced physiological response processes are trained using a combination of offline and online training. Offline training can use curated datasets to establish baseline model performance, while online training can allow the attention-induced physiological response processes to continually adapt and/or improve. The present disclosure recognizes the importance of maintaining strict data governance practices throughout this process to ensure user privacy is protected.

In some embodiments, the attention-induced physiological response processes may be designed with safeguards to maintain adherence to originally intended purposes, even as the attention-induced physiological response processes adapt based on new data. Any significant changes in data collection and/or applications of attention-induced physiological response process use may (and in some cases should) be transparently communicated to affected stakeholders and/or include obtaining user consent with respect to changes in how user data is collected and/or utilized.

Despite the foregoing, the present disclosure also contemplates embodiments in which users selectively restrict and/or block the use of and/or access to data. That is, the present disclosure contemplates that hardware and/or software elements can be provided to prevent or block access to data. For example, in the case of some services, the present technology should be configured to allow users to select to “opt in” or “opt out” of participation in the collection of data during registration for services or anytime thereafter. In another example, the present technology should be configured to allow users to select not to provide certain data for training the attention-induced physiological response processes and/or for use as input during the inference stage of such systems. In yet another example, the present technology should be configured to allow users to be able to select to limit the length of time data is maintained or entirely prohibit the use of their data for use by the attention-induced physiological response processes. In addition to providing “opt in” and “opt out” options, the present disclosure contemplates providing notifications relating to the access or use of personal information. For instance, a user can be notified when their data is being input into the attention-induced physiological response processes for training or inference purposes, and/or reminded when the attention-induced physiological response processes generate outputs or make decisions based on their data.

The present disclosure recognizes attention-induced physiological response processes should incorporate explicit restrictions and/or oversight to mitigate against risks that may be present even when such systems having been designed, developed, and/or operated according to industry best practices and standards. For example, outputs may be produced that could be considered erroneous, harmful, offensive, and/or biased; such outputs may not necessarily reflect the opinions or positions of the entities developing or deploying these systems. Furthermore, in some cases, references to or failures to cite third-party products and/or services in the outputs should not be construed as endorsements or affiliations by the entities providing the attention-induced physiological response processes. Generated content can be filtered for potentially inappropriate or dangerous material prior to being presented to users, while human oversight and/or ability to override or correct erroneous or undesirable outputs can be maintained as a failsafe.

The present disclosure further contemplates that users of the attention-induced physiological response processes should refrain from using the services in any manner that infringes upon, misappropriates, or violates the rights of any party. Furthermore, the attention-induced physiological response processes should not be used for any unlawful or illegal activity, nor to develop any application or use case that would commit or facilitate the commission of a crime, or other tortious, unlawful, or illegal act including misinformation, disinformation, misrepresentations (e.g., deepfakes), deception, impersonation, and propaganda. The attention-induced physiological response processes should not violate, misappropriate, or infringe any copyrights, trademarks, rights of privacy and publicity, trade secrets, patents, or other proprietary or legal rights of any party, and appropriately attribute content as required. Further, the attention-induced physiological response processes should not interfere with any security, digital signing, digital rights management, content protection, verification, or authentication mechanisms. The attention-induced physiological response processes should not misrepresent machine-generated outputs as being human-generated.

As described above, one aspect of the present technology is the gathering and use of physiological data to improve a user's experience of an electronic device with respect to interacting with electronic content. The present disclosure contemplates that in some instances, this gathered data may include personal information data that uniquely identifies a specific person or can be used to identify interests, traits, or tendencies of a specific person. Such personal information data can include physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve interaction and control capabilities of an electronic device. Accordingly, use of such personal information data enables calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information and/or physiological data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying and/or personal information about the user, such as a legal name, username, time and location data, or the like). In this way, other users, hackers, or third parties cannot determine the identity of the user associated with the stored data. In some implementations, a user may access his or her stored data from a user device that is different than the one used to upload the stored data. In these instances, the user may be required to provide login credentials to access their stored data.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various objects, these objects should not be limited by these terms. These terms are only used to distinguish one object from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “or” as used herein refers to and encompasses any and all possible combinations of one or more of the associated listed items. It will be further understood that the terms “comprises” or “comprising,” when used in this specification, specify the presence of stated features, integers, steps, operations, objects, or components, but do not preclude the presence or addition of one or more other features, integers, steps, operations, objects, components, or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

What is claimed is:

1. A method comprising:
at a device comprising a processor:
displaying, via a user interface, content that comprises an interaction element, wherein the interaction element comprises a plurality of regions, each region having different illumination characteristics;
providing instructions to focus on a particular region of the plurality of regions, the particular region being associated with a particular illumination characteristic;
obtaining a first set of physiological data for a first period of time, the physiological data associated with pupillary data; and
determining, based on the first set of physiological data, an attention-induced physiological response during presentation of the content for the first period of time corresponding to an interaction with the interaction element, wherein a process is configured to detect user interactions of a particular type based on detecting occurrences of attention-induced physiological responses by the user that are similar to the attention-induced physiological response.

2. The method of claim 1, further comprising:
updating a pupillary sensitivity level of the user interface based on the attention-induced physiological response.

3. The method of claim 1, further comprising:
obtaining a second set of physiological data for a second period of time, the physiological data associated with pupillary data;
determining, based on the second set of physiological data, a second attention-induced physiological response during presentation of the content for the second period of time corresponding to an interaction with the interaction element; and
updating the pupillary sensitivity level of the user interface based on the second attention-induced physiological response.

4. The method of claim 1, wherein the process is configured to detect user interactions of the particular type comprises predicting that the user is thinking of performing an act based on recognizing that a pattern exhibited in the pupillary data of the physiological data is indicative of the user thinking of performing the act.

5. The method of claim 1, further comprising:
determining user interaction feedback data corresponding to the interaction with the interaction element based on the attention-induced physiological response.

6. The method of claim 5, wherein the user interaction feedback data comprises providing instructions to the user via the user interface to initiate the attention-induced physiological response corresponding to an interaction event with the interaction element while thinking of performing an act.

7. The method of claim 1, wherein the interaction with the interaction element is classified using a machine learning technique based on the attention-induced physiological response and the different illumination characteristics of the regions.

8. The method of claim 1, wherein the different illumination characteristics of the interaction element comprises one or more dark regions and one or more bright regions.

9. The method of claim 8, wherein determining an attention-induced physiological response comprises:
determining scene-induced pupil response variation characteristics for the regions of the interaction element; and
determining the interaction event of the user during the presentation of content based on the scene-induced pupil response variation characteristics for the regions of the interaction element.

10. The method of claim 1, further comprising:
modifying the content via the user interface in response to determining the attention-induced physiological response corresponding to the interaction with the interaction element.

11. The method of claim 1, wherein the attention-induced physiological response comprises a pupillary response.

12. The method of claim 11, wherein the pupillary response is:
a direction of the pupillary response;
a velocity of the pupillary response; or
pupillary fixations.

13. The method of claim 1, wherein determining the attention-induced physiological response is based on a combination of neurological signals corresponding to a neurological response and pupillary data corresponding to a pupillary response.

14. The method of claim 1, wherein the device is a head-mounted device (HMD).

15. The method of claim 1, wherein the presentation of content is an extended reality (XR) experience that is presented to the user.

16. A device comprising:
a non-transitory computer-readable storage medium; and
one or more processors coupled to the non-transitory computer-readable storage medium, wherein the non-transitory computer-readable storage medium comprises program instructions that, when executed on the one or more processors, cause the one or more processors to perform operations comprising:
displaying, via a user interface, content that comprises an interaction element, wherein the interaction element comprises a plurality of regions, each region having different illumination characteristics;
providing instructions to focus on a particular region of the plurality of regions, the particular region being associated with a particular illumination characteristic;
obtaining a first set of physiological data for a first period of time, the physiological data associated with pupillary data; and
determining, based on the first set of physiological data, an attention-induced physiological response during presentation of the content for the first period of time corresponding to an interaction with the interaction element, wherein a process is configured to detect user interactions of a particular type based on detecting occurrences of attention-induced physiological responses by the user that are similar to the attention-induced physiological response.

17. The device of claim 16, wherein the non-transitory computer-readable storage medium comprises program instructions that, when executed on the one or more processors, further cause the one or more processors to perform operations comprising:
updating a pupillary sensitivity level of the user interface based on the attention-induced physiological response.

18. The device of claim 16, wherein the non-transitory computer-readable storage medium comprises program instructions that, when executed on the one or more processors, further cause the one or more processors to perform operations comprising:
obtaining a second set of physiological data for a second period of time, the physiological data associated with pupillary data;
determining, based on the second set of physiological data, a second attention-induced physiological response during presentation of the content for the second period of time corresponding to an interaction with the interaction element; and
updating the pupillary sensitivity level of the user interface based on the second attention-induced physiological response.

19. The device of claim 16, wherein the process is configured to detect user interactions of the particular type comprises predicting that the user is thinking of performing an act based on recognizing that a pattern exhibited in the pupillary data of the physiological data is indicative of the user thinking of performing the act.

20. A non-transitory computer-readable storage medium, storing program instructions executable by one or more processors on a device to perform operations comprising:
displaying, via a user interface, content that comprises an interaction element, wherein the interaction element comprises a plurality of regions, each region having different illumination characteristics;
providing instructions to focus on a particular region of the plurality of regions, the particular region being associated with a particular illumination characteristic;
obtaining a first set of physiological data for a first period of time, the physiological data associated with pupillary data; and
determining, based on the first set of physiological data, an attention-induced physiological response during presentation of the content for the first period of time corresponding to an interaction with the interaction element, wherein a process is configured to detect user interactions of a particular type based on detecting occurrences of attention-induced physiological responses by the user that are similar to the attention-induced physiological response.
